(19) United States
(12) Patent Application Publication
(10) Pub. No.: US 2006/0025729 A1
(43) Pub. Date: Feb. 2, 2006

(54) Transanal colorectal irrigators

(76) Inventors: Arnold R. Leiboff, Stony Brook, NY (US); Joseph Zipper, Miller Place, NY (US)

Correspondence Address: BRIAN ROFFE, ESQ, 11 SUNRISE PLAZA, SUITE 303, VALLEY STREAM, NY, 11580-6170, US

(21) Appl. No.: 11/191,833

(22) Filed: Jul. 28, 2005

Related U.S. Application Data
(60) Provisional application No. 60/592,888, filed on Jul. 30, 2004.
(60) Provisional application No. 60/662,402, filed on Mar. 16, 2005.

(51) Int. Cl. A61M 1/00

(52) U.S. Cl. 604/317

(57) ABSTRACT

Colorectal lavage device including a drain assembly, and a colonic tube assembly including an inflow tube, a flow control mechanism to control flow through the inflow tube, an outflow tube and a flow control mechanism to control flow through the outflow tube. The drain assembly has a tubular body defining an interior space and having a proximal portion insertable into an anus of a person to cause the anus to constrict around the proximal portion and thereby seal the proximal portion against the anal wall, a distal portion including a distal end having an opening in line with an opening in the proximal portion and a side tube arranged at an angle to the proximal portion. The inflow tube passes through the tubular body and has a tip with one or more apertures through which the lavage fluid operatively flows. The outflow tube is connected to the side tube.

### CROSS REFERENCE TO RELATED APPLICATIONS

This application claims priority under 35 U.S.C. 119(e) of U.S. provisional patent application Ser. No. 60/592,888 filed Jul. 30, 2004 and U.S. provisional patent application Ser. No. 60/662,402 filed Mar. 16, 2005, the specification of each of which is incorporated by reference herein.

### FIELD OF THE INVENTION

The present invention relates to devices for transanal lavage of the rectum and colon during a surgical operation.

The present invention also relates to irrigator devices for use in, for example, irrigating the rectum, to suction control devices for use in connection with suction tubes to control the flow of suction therethrough and to colonic tubes and assemblies including the same for use in irrigating the colon.

### BACKGROUND OF THE INVENTION

Surgical operations to remove the distal colon and part of the rectum are common for illnesses such as neoplasia, diverticular disease and inflammatory bowel disease. When these operations are performed it is beneficial to have the rectum and adjacent colon as clean of fecal matter as possible. Stool is composed mostly of bacteria, and removing stool minimizes the opportunity for bacterial contamination of the normally sterile body tissues (e.g., peritoneal cavity and layers of abdominal wall) and thereby minimizes the risk of postoperative infections (e.g., peritonitis or wound infection). Rectal lavage is also performed after removing a sigmoid or rectal cancer to help prevent cancer recurrence. Many surgeons prefer to lavage the isolated rectum with cytotoxic agents to remove or kill exfoliated tumor cells, which may implant and cause local recurrences, prior to performing an anastomosis to restore bowel continuity.

Several devices have been used previously to transanally introduce fluids into the rectum in order to cleanse the rectum, or rectum and adjacent colon, during surgery. In 1942, R. C. Chaffin patented a Surgical Suction Drainage and Irrigation Tube, U.S. Pat. No. 2,286,462. In 1958, Ryan introduced the use of a Chaffin sump, a bilumenal tube for intraoperative rectal lavage. In 1972, Alexander disclosed the use, for transanal colorectal lavage, of a No. 24 catheter, with several extra holes cut in the distal end, attached by way of a Y- connector both to an elevated bag of sterile saline solution and to a plastic drainage bag hung below the patient. In his book Surgery of the Anus, Rectum and Colon, Goligher describes his process of passing a catheter through an open proctoscope to wash the rectum below an occluding intestinal clamp. The disadvantage of this technique is that the washout fluid has to escape down waterproof sheeting to a receptacle at the foot of the table. In the same book, Goligher shows how he employs a 26 or 32 mm glass tube with side arm (after Muir) which he secures to the anus with a purse-string suture.

In the 1980s, Thow, inspired by the Chaffin sump used by Ryan, designed a biluminal tube which features large irrigation holes to facilitate cleansing and disinfection plus a 50 cc balloon to completely seal the rectum to prevent leakage and contamination of the surgical environment. This tube is commercially available as a component of the “Colo-Vage” intraoperative irrigation system, a complete colonic irrigation and drainage system which features a soft silicone rubber irrigation tube and a 7 foot fluid administration set with dual spikes and conveniently located “on-off” clamps (see hdccorp.com/surgical/SurgicalHome.html). In 1985, Scammell et al. published their technique of inserting a Foley catheter into the rectum, inflating the balloon and connecting the catheter to a funnel by tubing to facilitate the washout. In 1987, Goodson introduced an “irrigating proctoscope” consisting of a proctoscope with a specially designed cap which is fitted to the proctoscope with a rubber seal inside making it watertight when locked into position. The cap has two holes, a smaller one for receiving an irrigating tube, and a larger one for carrying away the fluid via a short metal spout with a long tube attached. The irrigating liquid was delivered by some source of positive pressure, such as a Higginson's syringe or a sheep drench gun.

In 1992, Infantino introduced his “Colo-Shower®” device for intraoperative rectal washout. This commercially available “sealed-circuit” system, in a sterile package, consists of a disposable modified anoscope, a watertight connector, and two tubes, one for irrigation and the other for collecting liquid flowing out of the anus (see sapimed.com/PRODUCTS.htm). After irrigation, the inner irrigating tube is removed and, to eliminate any residual fluid, the anoscope is withdrawn while connected via the collecting tube to an aspirator.

More recently, the Proctowash™ Rectal Washout Kit was introduced for sale in the U.S., after having been available in foreign markets for a limited time. This device is supplied by Intermark Medical Innovations LTD (UK) and is described in U.S. patent application 20050004533. It has a rigid probe through which is passed a fluid administration tube. Effluent drains through holes in the nozzle of the probe and down waste tubing to a waste container.

In the absence of these specialized devices, the surgeon may insert a rigid proctoscope, tilt the patient and angle the proctoscope so that the proctoscope's forward tip is dependently positioned, instill lavage fluid through the scope into the rectum using a syringe, and then aspirate with a suction cannula.

Numerous other devices have been patented for the administration of enemas or for colonic irrigation. The devices disclosed in the patents listed below, could also be adapted for intraoperative transanal rectal and colonic lavage: U.S. Pat. Nos. 397,060, 2,892,458, 2,936,760, 3,459,175, 3,053,257, 3,828,774, 3,842,834, 3,889,676, 4,019,515, 4,403,982, 4,406,655, 4,637,814, 4,712,536, 4,772,260, 4,842,580, 4,842,583, 4,874,363, 4,943,285, 4,986,822, 5,176,630, 5,351,674, 5,405,319, 6,106,506, 6,228,048 and 6,468,245.

Likewise, the devices disclosed in the published U.S. patent applications listed below, could be adapted for intraoperative rectal and colonic lavage: 20030114834, 20030195481, 20030073963, 20030073974 and 20040267198.

A disadvantage of the devices which employ an inflatable retention cuff (balloon) to prevent escape of lavage fluid and fecal matter around the catheter, is that these cuffs take time to inflate and deflate, prolonging and encumbering the lavage procedure. A further disadvantage of the inflatable retention cuffs is that they interfere with complete cleansing of the lower rectum and anus adjacent to and distal to the cuff. Furthermore, those catheters which employ retention cuffs have relatively small calibers, which impede the passage of large bits of feces.

A disadvantage of most of the non-cuffed tips is that their outer diameters are too small to prevent fluid from escaping around them if they were to be used in an anesthetized patient. In a non-anesthetized patient, the anal sphincter contracts around a tube to form a seal. In the anesthetized patient, the sphincter is relatively flaccid and will not seal well around narrow tubes. For intraoperative transanal colorectal lavage, a significantly wider tube is necessary to seal against the anal wall and prevent seepage around the tube.

### OBJECTS AND SUMMARY OF THE INVENTION

It is an object of the present invention to provide a device for intraoperative transanal rectal (or rectal and colonic) lavage which is more efficient than prior art devices.

It is another object of the present invention to provide a device for intraoperative transanal rectal (or rectal and colonic) lavage which is of sufficient caliber that it can reliably form a watertight seal where it abuts the inner anal wall of an anesthetized patient.

It is yet another object of the present invention to provide a device for intraoperative transanal rectal (or rectal and colonic) lavage which has an expanded portion which can form an additional seal when pressed against the anal opening.

It is still another object of the present invention to provide a device for intraoperative transanal rectal (or rectal and colonic) lavage which does not require an inflatable cuff (balloon) to prevent leakage of fecal matter around the device.

It is another object of the present invention to provide a sterile disposable sealed-circuit system for intraoperative rectal (or rectal and colonic) lavage which is simpler in design, has fewer parts, and is cheaper to manufacture than other devices.

It is yet another object of the present invention to provide an integral means to aspirate the lavage fluid and rectal waste directly into a standard disposable operating room suction cannister.

It is still another object of the present invention to provide a convenient means for the surgeon to control the vacuum pressure transmitted to the tip of an intraoperative transanal rectal (or rectal and colonic) lavage device while it is situated in the bowel.

It is still another object of the present invention to provide a convenient means to cleanse the rectum and distal colon intraoperatively, when the patient is in the supine position.

Another object of the present invention is to provide a suction control locator sleeve for use with a pinch clamp arranged on a suction tube having an aperture therein in order to control suction through the tube.

In order to achieve some of these objects and others, a rectal lavage device in accordance with the invention comprises an irrigator device including a tubular body defining an interior space and having a proximal portion adapted to be inserted into an anus of a person such that the proximal portion causes the anus to constrict around the proximal portion and thereby seal the proximal portion against the anal wall, a distal portion adapted to mate with inflow and outflow tubes, an expanded portion having a larger diameter than the proximal portion and interposed between the proximal portion and the distal portion, the expanded portion being adapted to engage with an anal opening to limit insertion of the proximal portion into the anus and seal the tubular body against the anal opening; an inflow tube; a flow control mechanism to control flow through the inflow tube; and an outflow tube. Some embodiments provide a flow control mechanism to control flow through the outflow tube. Some embodiments provide a large diameter outflow hose to facilitate straight drainage into a waste container by gravity. Other embodiments employ vacuum pressure to aspirate fecal effluent into a suction cannister waste receptacle. Some embodiments employ apertures in the tubular body and/or outflow tube which permit the control of vacuum pressure transmitted to the tubular body. The flow control mechanisms for the inflow tube and outflow tube may be clamps.

The tubular body may be dip molded in one piece so that it would be simple and inexpensive to manufacture and ideal for a single use, disposable product.

An exemplifying method for rectal lavage in accordance with the invention, using the device described above, includes the steps of inserting the proximal portion of the tubular body into the anus of a patient to cause the proximal portion to dilate the anus whereby the anus constricts around the proximal portion and a seal is formed between the proximal portion and the wall of the anus to prevent escape of fluid from the rectum, and the expanded portion is continuously pressed against the opening of the anus also to prevent the escape of fluid from the rectum. The inflow tube is adapted to a lavage fluid bag or container which is hung at an elevation above the patient's rectum, so that when the inflow tube flow control mechanism is opened, lavage fluid flows through the inflow tube, through the tubular body, into the rectum. When the rectum is filled, the inflow tube is closed, and the outflow tube flow control mechanism is opened to allow flow of waste from the rectum to the suction cannister waste receptacle. The level of vacuum pressure transmitted to the tubular body and rectum is controlled by incremental or complete occlusion of an aperture in the outflow tube by the surgeons finger. The lavage cycle is then repeated until the rectum is sufficiently clean.

An alternative embodiment is advantageous for irrigating both the rectum and a segment of colon of varying length proximal to the rectum. This embodiment comprises a tubular body defining an interior space and having a proximal portion adapted to be inserted into an anus of a person such that the proximal portion causes the anus to constrict around the proximal portion and thereby seal the proximal portion against the anal wall, a distal portion with two arms, one adapted to mate with an outflow tube, the other containing a constriction which will permit passage of an inflow tube through the tubular body while forming a seal around the inflow tube to prevent leakage of fluid around the inflow tube, an expanded portion having a larger size than the proximal portion and interposed between the proximal portion and the distal portion, the expanded portion being adapted to engage with an anal opening to limit insertion of the proximal portion into the anus and seal the tubular body against the anal opening; an aperture in the arm of the distal portion adapted to mate with the outflow tube which may be used to control the vacuum pressure transmitted to the tubular body; and an outflow tube. A separate inflow tube has a molded round tip with an aperture at one end, a spike adaptable to a fluid supply bag or container at the other, and a flow control mechanism, e.g., a clamp, to control flow through the inflow tube. The tubular body may be dip molded in one piece so that it would be simple and inexpensive to manufacture and ideal for a single use, disposable product.

An exemplifying method for rectal and colonic lavage in accordance with this alternative embodiment of the invention includes the steps of inserting the proximal portion of the body into the anus of a patient to cause the proximal portion to dilate the anus whereby the anus constricts around the proximal portion and a seal is formed between the proximal portion and the wall of the anus to prevent escape of air from the rectum, and the expanded portion is continuously pressed against the opening of the anus also to prevent the escape of fluid from the rectum. The inflow tube is passed through the inflow arm of the distal portion of the tubular body, through the body into the rectum, and through the rectum into the colon. A variable length of inflow tube may be passed through the colon, depending upon the length of colon to be irrigated. The inflow tube is adapted to a lavage fluid container which is hung at an elevation above the patient's rectum, so that when the inflow tube clamping means is opened, lavage fluid flows through the inflow tube into the colon, and then washes the colon and rectum. The fluid passes distally through the bowel around the inflow tube, into and through the tubular body and through the outflow tubing into the suction cannister waste receptacle. The level of vacuum pressure transmitted to the tubular body and rectum is controlled by incremental or complete occlusion of the aperture in the outflow arm of the distal portion of the tubular body by the surgeons finger. Lavage is continued until the colon and rectum are sufficiently clean.

Descriptions are provided of other embodiments which may more expeditiously remove residual effluent particularly when longer segments of colon are cleansed. Another embodiment is useful when the patient is supine.

### BRIEF DESCRIPTION OF THE DRAWINGS

The invention, together with further objects and advantages thereof, may best be understood by reference to the following description taken in conjunction with the accompanying drawings wherein like reference numerals identify like elements.

FIG. 1A is a top view of a first embodiment of a rectal irrigator in accordance with the invention.

FIG. 1B is a longitudinal cross-sectional view in the horizontal plane of the tubular body of the embodiment shown in FIG. 1A.

FIG. 1C is a rear view of the tubular body of the rectal irrigator shown in FIG. 1A.

FIG. 1D is a top view of the tubular body of the rectal irrigator seen in FIG. 1A showing another embodiment of the proximal portion of the tubular body.

FIG. 2A is a top view of a pinch clamp for use with the rectal irrigator shown in FIG. 1A.

FIG. 2B is a side view of the pinch clamp.

FIG. 3A is a top view of a suction control locator sleeve in accordance with the invention for use with the rectal irrigator shown in FIG. 1A.

FIG. 3B is a side cross-sectional view of the suction control locator sleeve.

FIG. 4A is a top view of a suction control locator sleeve around outflow tubing locked into a pinch clamp.

FIG. 4B is a side cross-sectional view of the suction control locator sleeve around the outflow tubing locked into the pinch clamp.

FIG. 5 is a diagram showing the use of the rectal irrigator of FIG. 1A inserted into the rectum of a patient for rectal lavage.

FIG. 6A is a top view of a second embodiment of a rectal irrigator.

FIG. 6B is a side view of the tubular body of the rectal irrigator shown in FIG. 6A.

FIG. 6C is a transverse cross-sectional view of the tubular body of the rectal irrigator shown in FIG. 6B through the plane marked 6C-6C.

FIG. 6D is a longitudinal cross-sectional view of the tubular body of the rectal irrigator shown in FIG. 6C through the plane marked 6D-6D.

FIG. 7 is a diagram showing the use of the rectal irrigator of FIG. 6A inserted into the rectum of a patient for rectal lavage.

FIG. 8A is a side view of a third embodiment of this invention, useful as a rectocolonic irrigator when the patient is supine.

FIG. 8B is a top view of the embodiment shown in FIG. 8A.

FIG. 8C is a longitudinal cross-sectional view of the embodiment shown in FIG. 8B through the plane marked 8C-8C.

FIG. 8D is a transverse cross-sectional view of the rectocolonic irrigator shown in FIG. 8C through the plane marked 8D-8D.

FIG. 9 is a diagram showing the use of the rectocolonic irrigator of FIG. 8A inserted into the rectum and sigmoid colon of a patient for rectosigmoid lavage.

FIG. 10A is a side view of a fourth embodiment of this invention with a wide diameter hose connecting the tubular body to a waste container.

FIG. 10B is a longitudinal cross-sectional view of the embodiment of a rectocolonic irrigator shown in FIG. 10A.

FIG. 10C is an isolated longitudinal cross-sectional view of a portion of the tubular body of the rectocolonic irrigator shown in FIG. 10A showing another embodiment of the distal end of the tubular body.

FIGS. 11A, 11B and 11C show different embodiments and means to connect a wide diameter outflow hose to a waste container.

FIG. 12 is a diagram showing the use of the rectocolonic irrigator of FIG. 10A inserted into the rectum and sigmoid colon of a patient for rectosigmoid lavage.

FIG. 13A is a side view of a fifth embodiment of this invention, useful as a rectoclonic irrigator.

FIG. 13B a longitudinal cross-sectional view of the embodiment of a rectocolonic irrigator shown in FIG. 13A.

FIG. 14A is a side view of a sixth embodiment of this invention, useful as a rectocolonic irrigator.

FIG. 14B is a longitudinal cross-sectional view of the embodiment of a rectocolonic irrigator shown in FIG. 14A.

FIG. 15A is a side view of a seventh embodiment of this invention, useful as a rectocolonic irrigator.

FIG. 15B is a longitudinal cross-sectional view of the embodiment of a rectocolonic irrigator shown in FIG. 15A.

FIG. 16A is a side view of an eighth embodiment of this invention, useful as a rectocolonic irrigator.

FIG. 16B is a longitudinal cross-sectional view of the embodiment of a rectocolonic irrigator shown in FIG. 16A.

FIG. 17 is a diagram showing the use of the rectocolonic irrigator of FIG. 16A inserted into the rectum and sigmoid colon of a patient for rectosigmoid lavage.

FIG. 18A is a side view of a ninth embodiment of this invention, useful as a rectocolonic irrigator.

FIG. 18B is a longitudinal cross-sectional view of the rectocolonic irrigator shown in FIG. 18A.

FIG. 19 is a diagram showing the use of the rectocolonic irrigator of FIG. 18A inserted into the rectum and sigmoid colon of a patient for rectosigmoid lavage.

FIG. 20A is a side view of a tenth embodiment of this invention, useful as a rectocolonic irrigator.

FIG. 20B is a longitudinal cross-sectional view of the rectocolonic irrigator shown in FIG. 20A

FIG. 21A is a side view of an eleventh embodiment of this invention, useful as a rectocolonic irrigator.

FIG. 21B is a longitudinal cross-sectional view of the rectocolonic irrigator shown in FIG. 21A.

FIG. 22A is a side view of a twelfth embodiment of this invention, useful as a rectocolonic irrigator.

FIG. 22B is a longitudinal cross-sectional view of the rectocolonic irrigator shown in FIG. 22A.

### DETAILED DESCRIPTION OF THE INVENTION

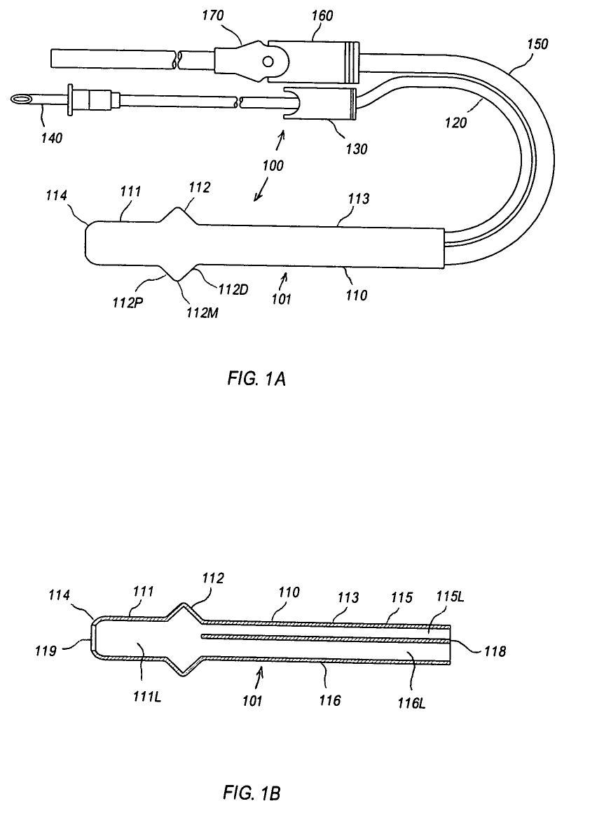

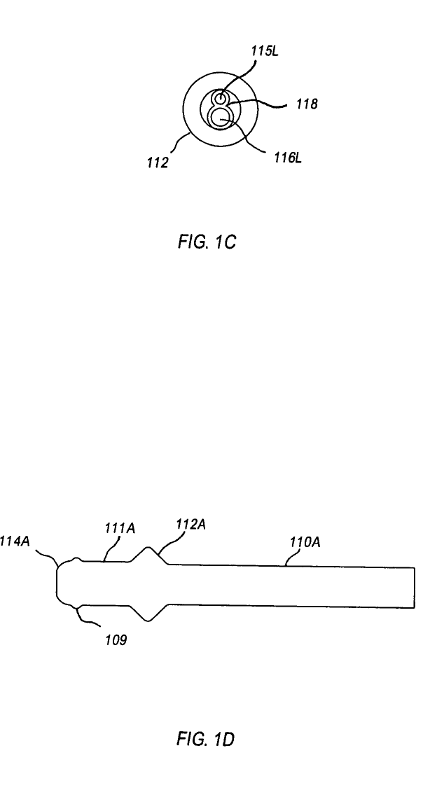

Referring to the accompanying drawings wherein like reference numerals refer to the same or similar elements, FIG. 1A shows a first embodiment of this invention, a rectal irrigator for rectal lavage in accordance with the invention which is designated generally as 100. Rectal irrigator 100 comprises an irrigator device 101 having a substantially tubular body 110, inflow tubing 120, a clamp 130 to control fluid inflow via inflow tubing 120, a spike 140 coupled to an inlet end of inflow tubing 120 and which adapts to a fluid container (not shown), outflow tubing 150, a clamp 160 to control outflow via outflow tubing 150 and a suction control locator sleeve 170.

Tubular body 110 comprises a proximal segment or portion 111 and a distal segment or portion 113 separated from the proximal portion 111 by an expanded segment or portion 112. Tubular body 110 of the rectal irrigator 100 may be made of a plastic or elastomer compatible for contact with internal parts of the human body, namely the anal wall and anal opening as discussed below, and fabricated using a dip molding technique to obtain a soft, pliable form. Other materials and manufacturing methods can also be used.

Proximal portion 111 has a substantially uniform inner and outer diameter with the exception that a tip 114 of the proximal portion 111 is rounded or tapered to aid insertion of proximal portion 111 into the anus of a patient, usually an anesthetized patient. An opening 119 is formed at the front end of the proximal portion 111.

Distal portion 113 has an inflow portion 115 defining an inflow lumen 115L, and an outflow portion 116 defining an outflow lumen 116L. Inflow portion 115 and outflow portion 116 share a common wall 118 and may be substantially parallel to one another as shown. Distal portion 113 has sufficient rigidity and length to function effectively as a handle for the tubular body 110. The outlet end of inflow tubing 120 inserts into the distal end of inflow portion 115 and the inlet end of outflow tubing 150 inserts into the distal end of outflow portion 116. The cross-sectional shape of inflow lumen 115L and outflow lumen 116L of tubular body 110 are therefore designed to correspond to the cross-sectional shape of inflow tubing 120 and outflow tubing 150, respectively.

Expanded portion 112 has an outer circumferential portion 112M having the largest diameter of the entire tubular body 110 and which is dimensioned to be larger than the opening of most human anuses when they are maximally dilated so that the tubular body 110 cannot be inserted into the anus farther than the outer circumferential portion 112M. That is, the expanded portion 112 serves as insertion-limiting flange which serves to limit the amount of insertion of the tubular body 110 into the anus. The expanded portion 112 includes two truncated conical surfaces 112P, 112D, one surface 112P tapering from the outer circumferential portion 112M toward the rear edge of the proximal portion 111 and one surface 112D tapering from the outer circumferential portion 112M toward the front edge of the distal portion 113.

FIG. 1D shows tubular body 110A having another embodiment of the proximal portion 111A of the rectal irrigator shown in FIG. 1A. Proximal portion 111A has circumferential lip 109, which is a second expanded portion of lesser diameter than expanded portion 112A. When tip 114A of tubular body 110A is seated in the rectum, the anus constricts around proximal portion 111A between lip 109 and expanded portion 112A. Lip 109 serves to retain proximal portion 11A of tubular body 110A in the anorectum, and helps to prevent inadvertent expulsion of the device.

Lip 109 can be provided on the tubular body of all of the embodiments shown herein.

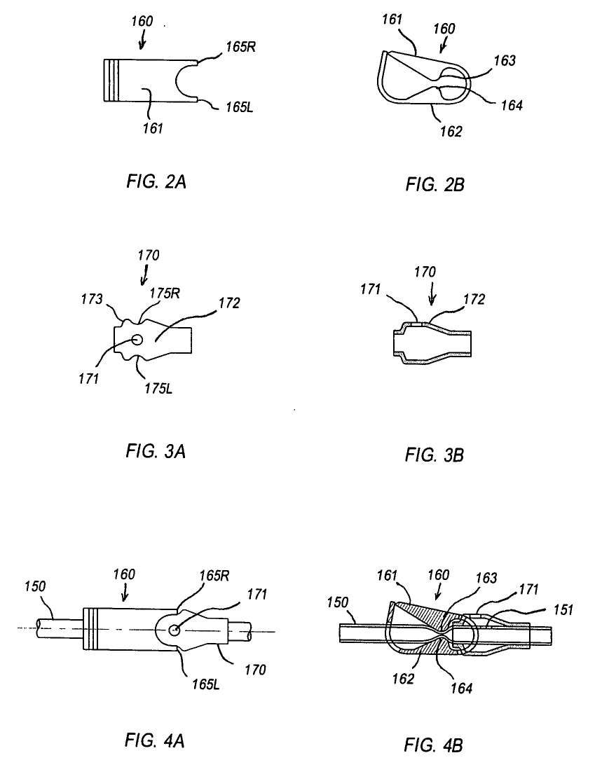

Clamps, preferably of the form depicted in FIGS. 2A and 2B, are used to control flow into and out of the rectum. Pinch clamp 130 controls flow of lavage fluid from the fluid container into tubular body 110 and the rectum by pinching or releasing the inflow tubing 120. Pinch clamp 160 controls the flow of lavage fluid and fecal waste from the rectum and tubular body 110 to the waste receptacle (not shown) via the outflow tubing 150. Pinch clamp 160 occludes the outflow tubing 150 when the surgeon compresses pinch clamp 160 by squeezing the top surface 161 with his thumb toward the bottom surface 162. The outflow tubing 150 is compressed and occluded between the two pinch surfaces 163 and 164.

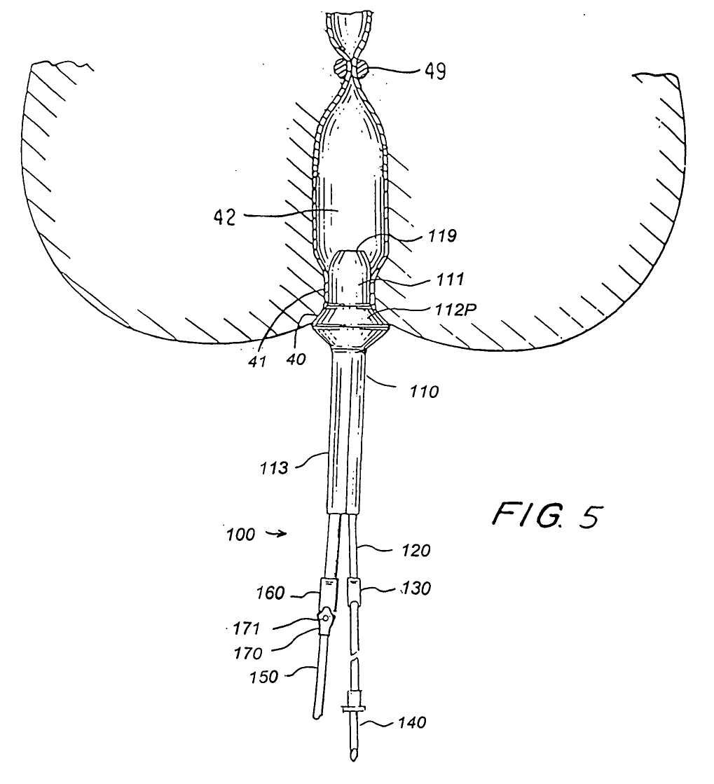

FIGS. 3A and 3B show suction control locator sleeve 170 in accordance with the invention that is used to control vacuum pressure transmitted to tubular body 110, and rectum when the rectal irrigator 100 is being used, via the outflow tubing 150. Suction control locator sleeve 170 has an aperture 171 in its top surface 172 and defines a channel through which the outflow tubing 150 passes. Suction control locator sleeve 170 is mounted on outflow tubing 150 so that suction control locator sleeve aperture 171 is directly, or nearly directly, over an outflow tubing aperture 151, as shown in FIG. 4B, or in another position in flow communication with and fixed relative to the outflow tubing aperture 151. Occlusion of suction control locator sleeve aperture 171 by the surgeon's thumb or another finger prevents ambient air from entering outflow tubing 150 via apertures 171 and 151, effectively increasing the vacuum pressure transmitted to tubular body 110 and the rectum. On the other hand, the absence of the surgeon's thumb or another finger over suction control locator sleeve aperture 171 enables ambient air flow into outflow tubing 150 via apertures 151, 171, effectively decreasing the vacuum pressure transmitted to tubular body 110.

Referring to FIG. 3A, suction control locator sleeve 170 has indentations 175L and 175R on either side. Indentations 175L and 175R permit struts 165L and 165R of pinch clamp 160 to retain a forward end 173 of suction control locator sleeve 170 inside pinch clamp 160 (see FIGS. 4A and 4B). This joins suction control locator sleeve 170 to pinch clamp 160 and prevents rotation of suction control locator sleeve 170 relative to pinch clamp 160, thereby maintaining aperture 171 in close proximity to and in alignment with the top surface 161 of pinch clamp 160. This allows the surgeon to quickly and ergonomically sequentially operate pinch clamp 160 and occlude the suction control locator sleeve aperture 171, and do so repetitively without having to release outflow tubing 150 from his hand or search for aperture 171.

Referring now to FIG. 5, in one exemplifying use of the rectal irrigator 100 to lavage the rectum, the proximal portion 111 of tubular body 110 of rectal irrigator 100 is inserted through the anus 41 of an anesthetized patient into the rectum 42. The surgeon holds tubular body 110 by grabbing distal portion 113 with one hand and presses surface 112P of expanded portion 112 of tubular body 110 against the anal opening 40. Spike 140 is inserted into the spike adapter of a fluid container. The end of outflow tubing 150 is adapted to a suction cannister waste receptacle. With outflow tubing pinch clamp 160 closed, inflow tubing pinch clamp 130 is opened to allow lavage fluid to flow from the fluid container through inflow tubing 120, into tubular body 110 and into the rectum 42. The lavage fluid fills the rectum 42 to where the rectum 42 has been occluded by the surgeon with a surgical clamp 49. Fluid cannot leak from the anus 41 because the proximal portion 111 of tubular body 110 is dimensioned such that its walls dilate the anus 41 whereby the elastic anus constricts around proximal portion 111 of tubular body 110 to form a seal between the walls of proximal portion 111 and the wall of the anus 41. Thus, fluid cannot pass between the outer wall of tubular body 110 and the wall of the anus 41. Surface 112P of expanded portion 112 is pressed against the anal opening 40 to form an additional seal.

When sufficient fluid has been infused and the rectum is distended, inflow tubing pinch clamp 130 is closed and outflow tubing pinch clamp is opened. Lavage fluid and fecal waste flow from the rectum 42 through opening 119 into tubular body 110, into the outflow tubing 150 and is aspirated into the suction cannister waste receptacle. The cycle of filling and emptying the rectum 42 can then be repeated until the rectum is sufficiently clean.

When aperture 171 of suction control locator sleeve 170 is open, ambient air is aspirated through the suction control locator sleeve aperture 171 and outflow tubing aperture 151 into outflow tubing 150, thereby limiting the vacuum pressure transmitted to tubular body 110 and the rectum 42. Lavage effluent and fecal waste are forced into and through tubular body 110 and into the outflow tubing 150 primarily by gravity and the elevated pressure in the rectum 42. Transmitting vacuum pressure to the rectum 42 is not desired at this point, because the vacuum pressure could result in rectal tissue being sucked into opening 119 of tubular body 110 thereby occluding the opening 119, interfering with rectal emptying. However, when the rectum 42 has largely emptied, there is no longer pressure on the fluid in the rectum 42. Fluid pressure and gravity are not able to empty all residual fluid and fecal waste. Now it is useful to occlude the suction control locator sleeve aperture 171 to allow vacuum pressure to be transmitted to tubular body 110 and the rectum 42 so that residual fluid can be aspirated.

In one exemplifying embodiment, the outer diameter of the walls defining the proximal portion 111 is approximately 1.062 inches, which has been determined to be suitable to allow it to be inserted into the anal canal of an anesthetized patient with ease and at the same time, prevent the escape of fluid from the rectum 42 by passing between the wall of tubular body 110 and wall of the anus 40. The largest diameter of the expanded portion 112, i.e., the outer circumferential portion 112M, has an approximate outer diameter of 1.75 inches which is too large to enter the anal canal (without applying excessive force) and which abuts and occludes the anal opening 40 when the proximal portion 111 is inserted completely into the anus 41. The expanded portion 112 thus serves to both limit the extent to which tubular body 110 is inserted into the rectum 42 and occlude the anal opening 40 to prevent leakage of fluid around tubular body 110.

These dimensions, while allowing for optimal performance, also permit tubular body 110 to be formed by a dip molding process, which is less expensive than other molding processes (e.g., injection molding). If the diameter of proximal portion 111 were appreciably smaller, or if the maximum diameter 112M of the expanded portion 112 were appreciably larger, the mandrel, around which the device is molded could not be readily removed from the device (unless the walls of the device were made thinner or more elastic, which would render the device too flimsy for use).

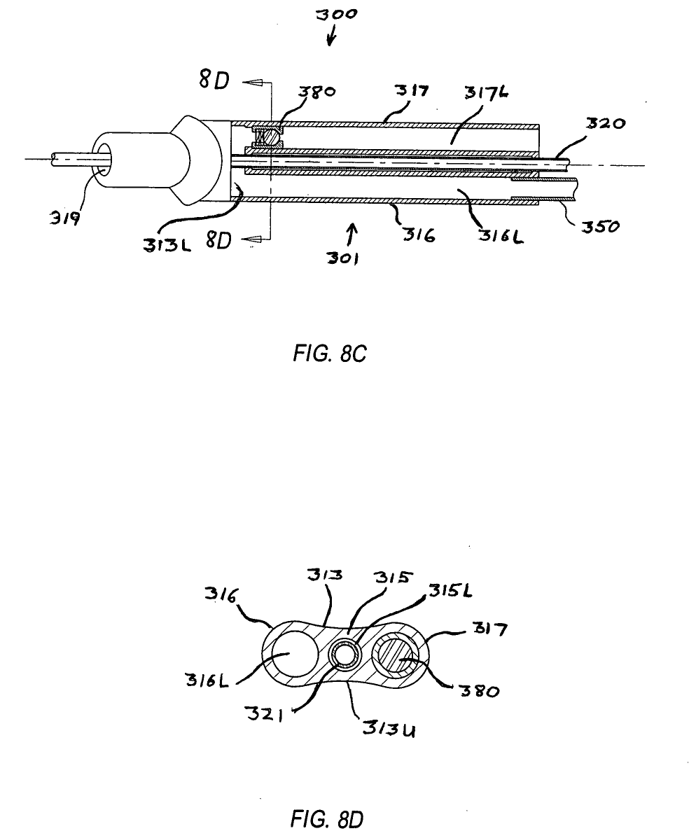

In a modified embodiment (not illustrated), distal portion 113 may be angled so that it is approximately 45 degrees to the co-axial axes of proximal portion 111 and expanded portion 112 (in a similar manner as shown and discussed with reference to FIGS. 8A-8D below). This arrangement permits tubular body 110 to lie flat on the surface of the operating room table, while proximal portion 111 projects directly in the anus and rectum of a patient lying in the supine position.

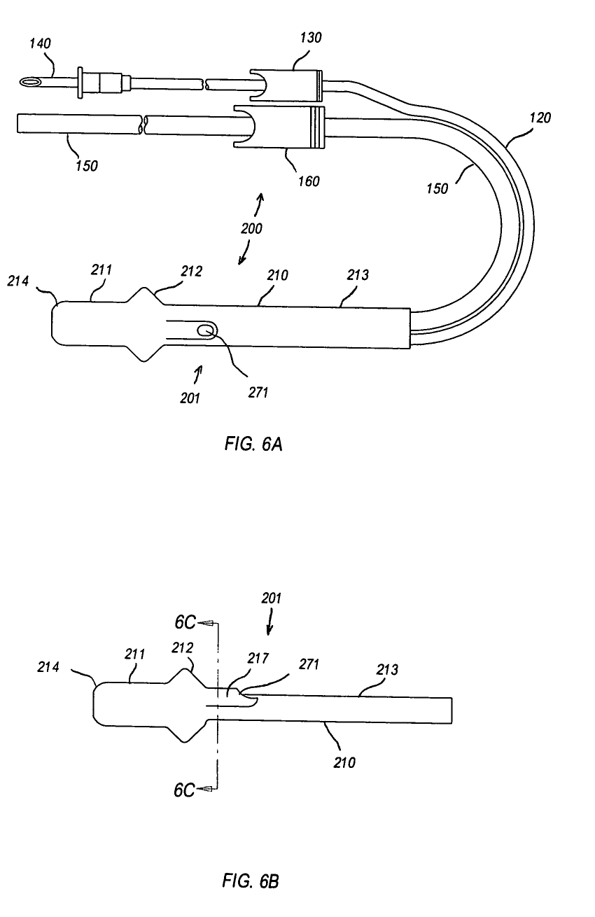

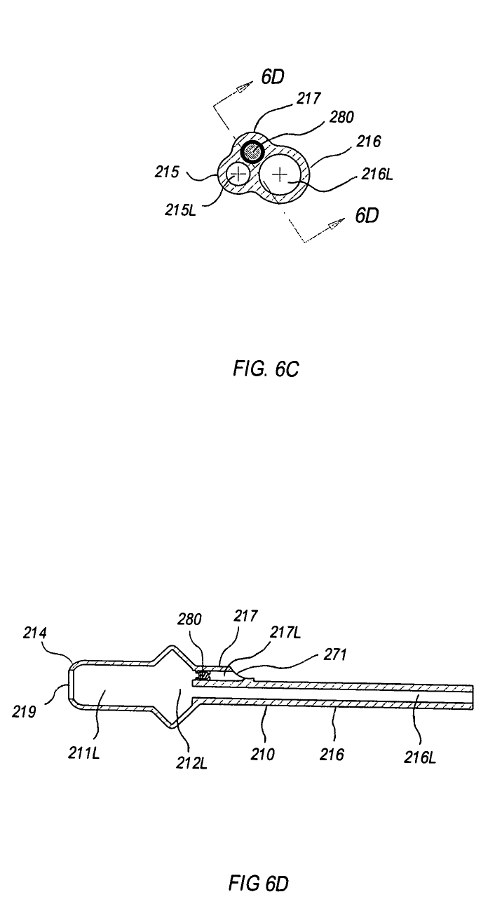

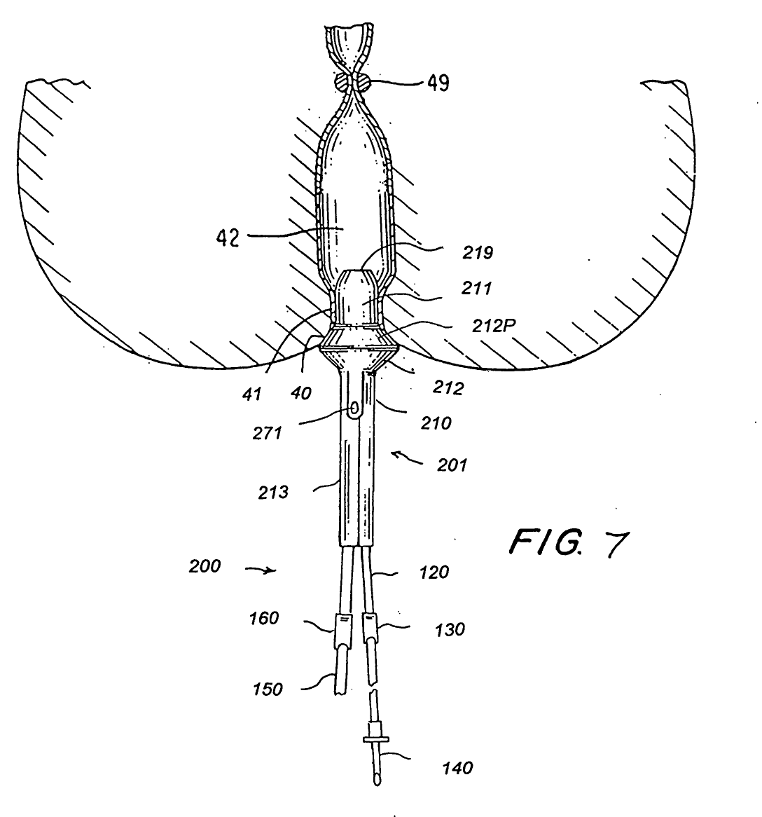

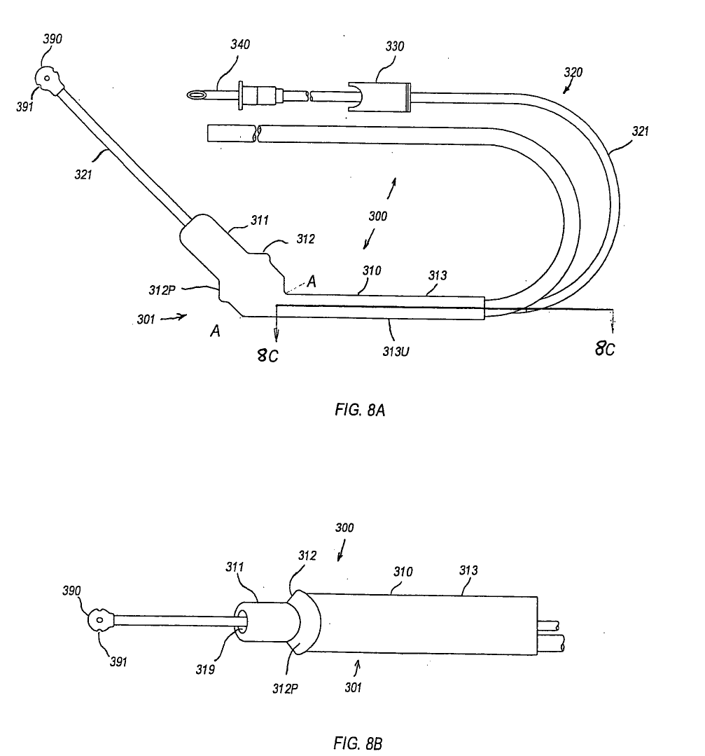

Referring now to FIGS. 6A, 6B, 6C and 6D, a second embodiment of the invention, a rectal irrigator for rectal lavage, is shown in its entirety in FIG. 6A and is designated generally as 200. Rectal irrigator 200 comprises essentially the same tubing and associated flow control devices as rectal irrigator 100, i.e., inflow tubing 120, clamp 130, spike 140, outflow tubing 150 and clamp 160, and a different irrigator device 201.

Irrigator device 201 includes a substantially tubular body 210 and a check valve 280 arranged in connection with tubular body 210.

Tubular body 210 comprises a proximal segment or portion 211 and a distal segment or portion 213 separated from the proximal portion 211 by an expanded segment or portion 212. Tubular body 210 of irrigator device 201 may be made of a plastic or elastomer compatible for contact with internal parts of the human body, namely the anal wall and anal opening as discussed below, and fabricated using a dip molding technique to obtain a soft, pliable form. Other materials and manufacturing methods can also be used.

Proximal portion 211 and expanded portion 212 have forms, dimensions, constructions, and purposes which are identical to corresponding portions 111 and 112 of tubular body 110. The proximal portion 211 can also have the form of proximal portion 11A of tubular body 110A shown in FIG. 1D.

Distal portion 213 has an inflow portion 215 defining a fluid inflow lumen 215L, an outflow portion 216 defining an outflow lumen 216L and air inflow portion 217 defining air inflow lumen 217L (see FIGS. 6C and 6D). Distal portion 213 has sufficient rigidity and length to function effectively as a handle for tubular body 210.

The outlet end of inflow tubing 120 inserts into the distal end of inflow portion 215 and the inlet end of outflow tubing 150 inserts into the distal end of outflow portion 216. The cross-sectional shape of inflow lumen 215L and outflow lumen 216L of tubular body 210 are therefore designed to correspond to the cross-sectional shape of inflow tubing 120 and outflow tubing 150, respectively.

Air inflow portion 217 contains check valve 280, which permits ambient air to be sucked through a suction control aperture 271, through check valve 280 and into an interior space 212L, defined by expanded portion 212, when suction control aperture 271 is not occluded.

Suction control aperture 271 in air inflow portion 217 is used to control vacuum pressure transmitted to interior space 211L, defined by proximal portion 211 of tubular body 210, and to the rectum via the outflow tubing 150. Occlusion of suction control aperture 271 by the surgeon's thumb or another finger prevents ambient air from entering lumen 217L, which effectively increases the vacuum pressure transmitted to interior space 211L and the rectum. On the other hand, the absence of the surgeon's thumb or another finger over suction control aperture 271 enables ambient air flow into air inflow lumen 217L and then into interior space 212L via check valve 280, effectively decreasing the vacuum pressure transmitted to interior space 211L.

Thus, instead of controlling the suction at a location along the outflow tubing as in the embodiment shown in FIG. 1, in this embodiment, suction is controlled at a location on the tubular body. Control of the suction in both cases involves the surgeon placing his thumb or another finger over a suction control aperture to prevent sucking of ambient air into the device.

Referring now to FIG. 7, in one exemplifying use of rectal irrigator 200 to lavage the rectum, proximal portion 211 of tubular body 210 of rectal irrigator 200 is inserted through the anus 41 of an anesthetized patient into the rectum 42. The surgeon holds tubular body 210 by grabbing distal portion 213 with one hand and presses surface 212P of expanded portion 212 of tubular body 210 against the anal opening 40. Spike 140 is inserted into the spike adapter of a fluid container (not shown). The outlet end of outflow tubing 150 is adapted to a suction cannister waste receptacle. With outflow tubing pinch clamp 160 closed, inflow tubing pinch clamp 130 is opened to allow lavage fluid to flow from the fluid container through inflow tubing 120, into tubular body 210 and into the rectum 42. The lavage fluid fills the rectum 42 to where the rectum 42 has been occluded by the surgeon with surgical clamp 49. Check valve 280 prevents fluid from escaping through air inflow lumen 217L of air inflow portion 217. Fluid cannot escape from the anus 41 because the proximal portion 211 of tubular body 210 is dimensioned such that its walls dilate the anus 41 whereby the elastic anus 41 constricts around proximal portion 211 of tubular body 210 to form a seal between the walls of proximal portion 211 and the wall of the anus 41. Thus, fluid cannot pass between the outer wall of tubular body 210 and the wall of the anus 41. Surface 212P of expanded portion 212 is pressed against the anal opening 40 to form an additional seal.

When sufficient fluid has been infused and the rectum 42 is distended, inflow tubing pinch clamp 130 is closed and outflow tubing pinch clamp 160 is opened. Lavage fluid and fecal waste flow from the rectum 42 into tubular body 210, into outflow tubing 150 and is aspirated into the suction cannister waste receptacle. The cycle of filling and emptying the rectum 42 can be repeated until the rectum 42 is sufficiently clean.

When suction control aperture 271 is open (uncovered), ambient air is aspirated through suction control aperture 271 into air inflow lumen 217A, through check valve 280, into interior space 212L of tubular body 210, and through outflow tubing 150, thereby limiting the vacuum pressure transmitted to interior space 211L of tubular body 210 and the rectum 42. Lavage effluent and fecal waste flow into tubular body 210 primarily by the force of gravity and the elevated pressure in the rectum 42. Transmitting vacuum pressure to the rectum 42 is not desired at this point, because the vacuum pressure could result in rectal tissue being sucked into opening 219 of tubular body 210, thereby occluding the opening and interfering with rectal emptying. However, when the rectum 42 has largely emptied, there is no longer pressure on the fluid in the rectum 42. Fluid pressure and gravity are not able to empty all residual fluid and fecal waste from the rectum 42. Now, it is useful to occlude suction control aperture 271 with a thumb or another finger to allow increased vacuum pressure to be transmitted to interior space 211L of tubular body 210 and the rectum 42 so that residual fluid can be aspirated.

The foregoing embodiments are all useful when the patient is operated upon in the modified lithotomy position, where access to the perineum and anus is available between the patient's legs, which are supported by stirrups. The tubular bodies of those embodiments are designed to be held in a surgeon's hand, or in the hand of a surgical assistant, so that the proximal portion sits in the anus and the expanded portion is pressed against the anal opening. However, surgeons often perform colon surgery while the patient is in the supine position, and access to the perineum and anus is not available.

Referring now to FIGS. 8A, 8B, 8C and 8D, a third embodiment of the invention, referred to as a rectocolonic irrigator, is useful when the patient is supine on the operating room table and when bowel to be lavaged includes a segment of sigmoid colon in addition to the rectum, and is shown in its entirety in FIG. 8A and designated generally as 300. Rectocolonic irrigator 300 comprises a drain assembly 301 having an angled tubular body 310, check valve 380 and outflow tubing 350, and colonic tube assembly 320.

Tubular body 310 comprises proximal portion 311 and distal portion 313 separated from the proximal portion 311 by expanded portion 312. Tubular body 310 may be made of a plastic or elastomer compatible for contact with internal parts of the human body, namely the anal wall and anal opening, and fabricated using a dip molding technique to obtain a soft, pliable form. Other materials and manufacturing methods can also be used.

Proximal portion 311 and expanded portion 312 have forms, dimensions, constructions, and functions identical to corresponding proximal and expanded portions 111 and 112 of rectal irrigator 100, respectively.

Distal portion 313 has inflow portion 315 defining fluid inflow lumen 315L, outflow portion 316 defining outflow lumen 316L and air inflow portion 317 defining air inflow lumen 317L. In this embodiment, all three portions, 315, 316 and 317, are arranged in the horizontal plane, so that distal portion 313 has a relatively flat undersurface 313U. Distal portion 313 is angled at line A-A so that it is approximately 45 degrees to the co-axial axes of proximal portion 311 and expanded portion 312. This arrangement permits tubular body 310 to lie flat on the surface of the operating room table, while proximal portion 311 projects directly into the anus and rectum of the patient. Tubular body 310 thereby lies in a stable disposition, fostering retention and resisting accidental dislodgement, without the need for an inflatable retention cuff, as is incorporated in other devices also useful when the patient is supine, such as a Foley catheter or the Colo-Vage intra-operative irrigation system.

Colonic tube assembly 320 comprises tip 390, inflow tube 321, inflow pinch clamp 330 arranged on inflow tube 321 and spike 340 attached to an inlet end of the inflow tube 321 and which adapts to a fluid container (not shown).

Inflow tube 321 traverses tubular body 310 through inflow portion 315, expanded portion 312 and proximal portion 311 to project into the patient's colon. Inflow tube 321 has a bulbous tip 390 which has apertures 391. Inflow tube 321 may be movable within body 310 to vary the distance between the tip 390 and the body 310.

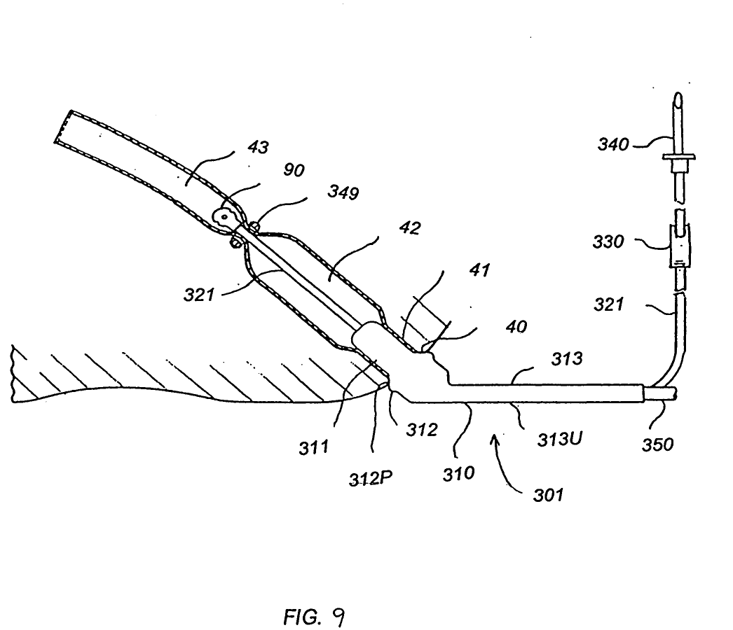

Pinch clamp 330 controls the flow of lavage fluid from the fluid container through inflow tube 321 into the colon during use (discussed below with reference to FIG. 9).

Outflow tubing 350 inserts into the distal end of outflow portion 316.

Air inflow portion 317 contains check valve 380, which permits ambient air to be sucked through the open distal end of air inflow portion 317, through check valve 380 and into interior space 313L (see FIG. 8C). Air is then sucked into outflow portion 316 and through outflow tubing 350. This air flow assists in flushing effluent and fecal waste which enters tubular body 310 through opening 319 out of the tubular body 310, through outflow tubing 350 and into the suction cannister waste receptacle. This air flow also limits the vacuum pressure transmitted to the interior of proximal portion 311 and to the rectum, and thereby prevents the rectum from being sucked into and occluding opening 319.

Referring now to FIG. 9, in one exemplifying use to lavage the colon and rectum proximal portion 311 of tubular body 310 of rectocolonic irrigator 300 is inserted through the anus 41 of a supine anesthetized patient into the rectum 42. Distal portion 313 lies flat so that undersurface 313U is in contact with the operating table. Tubular body 310 thereby rests in a stable disposition, unlikely to accidently dislodge from the anorectum. Spike 340 is inserted into the spike adapter of a fluid container (not shown). The end of outflow tubing 350 is adapted to a suction cannister waste receptacle. The surgeon cross-clamps the bowel 43 just below bulbous tip 390 with an atraumatic bowel occluding clamp 349, or with a Cooley Caval Occlusion Clamp, without compressing inflow tube 321. Inflow tube pinch clamp 330 is opened to allow lavage fluid to flow from the fluid container through inflow tube 321 and into the colon 43. The lavage fluid fills the colon 43 above the bowel occluding clamp 349. When the segment of colon 43 is sufficiently filled with fluid, bowel occluding clamp 349 is removed, and effluent and fecal waste flow distally in the colon 43, around the inflow tube 321, through the rectum 42, through opening 319 into tubular body 310, through outflow portion 316 and outflow tubing 350 into the suction cannister waste receptacle. Check valve 380 prevents fluid from escaping tubular body 310 through lumen 317L. Fluid cannot escape from the anus 41 because the proximal portion 311 of tubular body 310 is dimensioned such that its walls dilate the anus 41 whereby the elastic anus constricts around proximal portion 311 of tubular body 310 to form a seal between the walls of proximal portion 311 and the wall of the anus 41. Thus, fluid cannot pass between the outer wall of tubular body 310 and the wall of the anus 41. As the patient lies on tubular body 310, surface 312P of expanded portion 312 of tubular body 310 is pressed against anal opening 40 to form an additional seal.

The cycle of filling and drainage is repeated until the colon and rectum are sufficiently clean.

In a modified embodiment (not shown), inflow tube 321 does not pass through tubular body 310, but rather the outlet end of inflow tube 321 joins portion 315 of tubular body 310 at the distal end of distal portion 313 of tubular body 310. Bulbous tip 390 is eliminated. The device functions similarly to rectal irrigator 200 except that since the patient is supine, the surgeon does not have access to the tubular body, and cannot occlude the air inflow lumen to increase suction pressure. Also, like rectal irrigator 200, this modified embodiment employs a pinch clamp on the outflow tubing and the surgeon is not required to clamp across the bowel and tubing beneath bulbous tip 390 to lavage bowel proximal to the device. However, this outflow tubing pinch clamp must be situated some distance from the tubular body so that it can be accessed by operating room personnel when lavage fluid is to be infused into the bowel.

Subsequent embodiments to be described, i.e., embodiments four through twelve, are also useful when bowel to be lavaged includes colon in addition to rectum, and are therefore referred to as rectocolonic irrigators. However, like the first and second embodiments, embodiments four through twelve are used when the patient is in the modified lithotomy position, where the surgeon or assistant has access to the perineum.

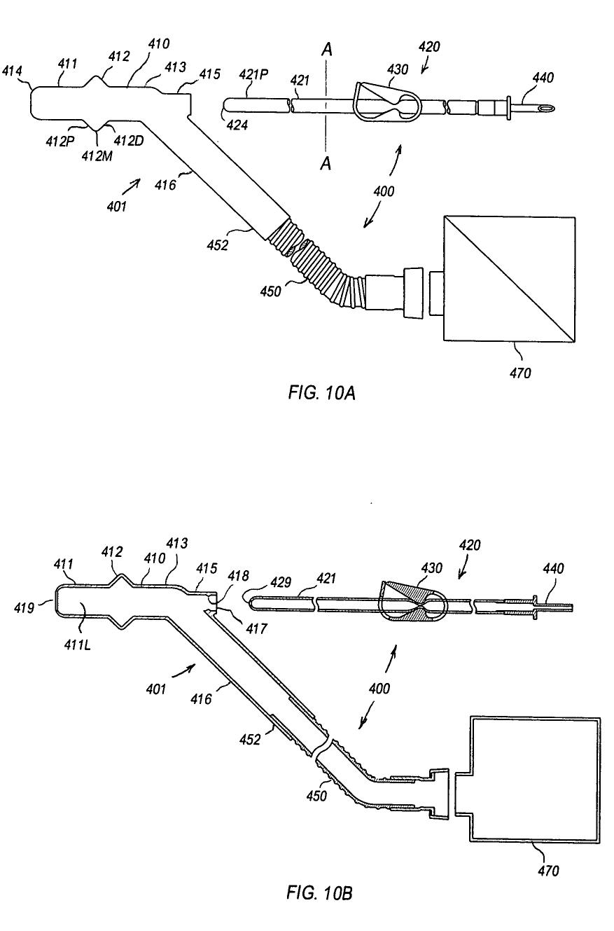

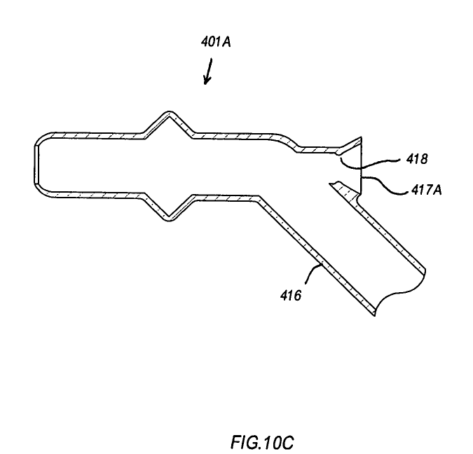

FIGS. 10A and 10B illustrate a fourth embodiment of the invention, rectocolonic irrigator 400, which comprises a drain assembly 401 and a colonic tube assembly 420.

Drain assembly 401 comprises tubular body 410 and outflow tubing 450, which is connected to the outlet 452 of side tube 416 of tubular body 410. Outflow hose 450 is a wide or large diameter outflow hose.

Tubular body 410 has a proximal segment or portion 411, which defines an interior space 411L, and a distal segment or portion 413 separated from proximal portion 411 by an expanded segment or portion 412. Tubular body 410 of rectocolonic irrigator 400 may be made of an elastomer compatible for contact with internal parts of the human body, namely the anal wall and anal opening as discussed below, and fabricated using a dip molding technique to obtain a soft, pliable form. Other materials and manufacturing methods can also be used.

Proximal portion 411, which defines an interior space 411L, has a substantially uniform inner and outer diameter with the exception that a tip 414 of the proximal portion 411 is rounded or tapered to aid insertion of proximal portion 411 into the anus of an anesthetized patient. An opening 419 is formed at the front end of proximal portion 411 (see FIG. 10B). The proximal portion 411 can also have the form of proximal portion 11A of tubular body 10A, shown in FIG. 1D.

Expanded portion 412 has an outer circumferential portion 412M having the largest diameter of the entire tubular body 410 and which is dimensioned to be larger than the opening of most human anuses when they are maximally dilated so that tubular body 410 cannot be inserted into the anus farther than the outer circumferential portion 412M. That is, the expanded portion 412 serves as insertion-limiting flange which serves to limit the amount of insertion of the proximal portion 411 of tubular body 410 into the anus. The expanded portion 412 includes two truncated conical surfaces 412P, 412D, one surface 412P tapering from the outer circumferential portion 412M toward the rear edge of the proximal portion 411 and one surface 412D tapering from the outer circumferential portion 412M toward the front edge of the distal portion 413.

Distal portion 413 has distal end 415 and a side tube 416 extending at an angle to proximal portion 411.

Distal end 415 is adapted to operatively receive a colonic tube 421 of colonic tube assembly 420. Colonic tube 421 may pass through a distal opening 417 into and through tubular body 410 and through the rectum into the sigmoid colon. Distal end 415 has an annular narrowing 418 which functions as a seal to prevent fluid from escaping through distal end 415 around colonic tube 421. Distal opening 417 may be cylindrical, which aids in demolding, or conical (see distal opening 417A in drain assembly 401A as shown in FIG. 10C), which facilitates insertion of colonic tube 421 into tubular body 410. The annular seal 418 may be formed integral with tubular body 410.

Distal end 452 of side tube 416 of distal portion 413 is joined to outflow hose 450.

Colonic tube assembly 420 comprises colonic tube 421, a clamp 430 to control fluid inflow via colonic tube 421 and a spike 440 coupled to an inlet end of colonic tube 421 and which adapts to a fluid container, such as a lavage fluid container (not shown). It is possible to provide the colonic tube 421 with a plurality of arms, in which case, a spike is attached to an inlet end of each arm of the colonic tube for connecting that arm to a respective fluid container. Instead of using spikes, an inlet end of the colonic tube 421 may also be integrally attached to a lavage fluid container, and if the colonic tube has a plurality of arms, each arm can be integrally connected to a respective fluid container.

Colonic tube 421 is an elongated tube having spike 440 at its inlet end and aperture 429 at its tip 424 at the outlet end. One or more additional apertures may be placed in the wall of colonic tube 421 in proximity to tip 424. Colonic tube 421 may be fabricated from an elastomeric material compatible for contact with internal parts of the human body, namely the bowel.

Clamp 430, preferably of the form depicted in FIGS. 2A and 2B, controls flow of lavage fluid from the fluid container through the colonic tube 421 into the bowel by pinching or releasing colonic tube 421.

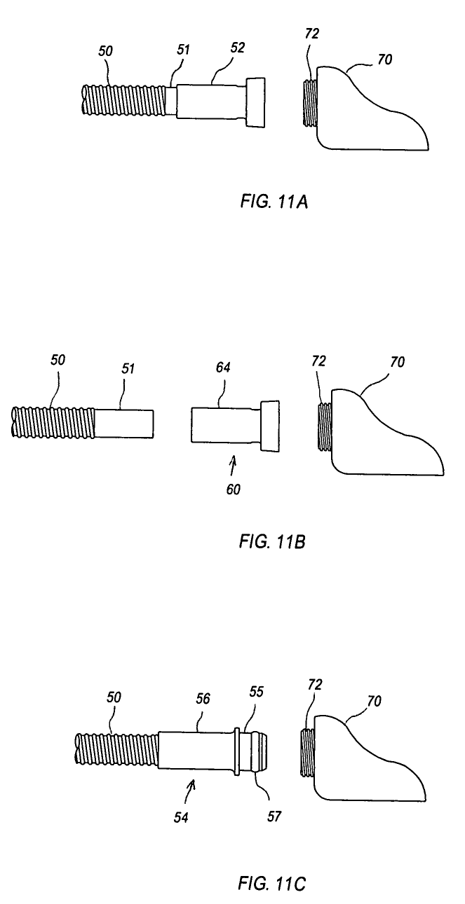

Referring now to FIGS. 11A, 11B and 11C, three different means to form a connection of a large diameter outflow hose 50 to a waste container 70 are shown (and this connection may be used to connect large diameter outflow hose 450 or a large diameter outflow hose described in another embodiment herein to a waste container). FIG. 11A shows an end piece 52 bonded to an outlet end 51 of outflow hose 50. End piece 52 contains a perforated cap 53 which removably screws onto a threaded inlet 72 of waste container 70. FIG. 11B shows outflow hose 50 having an end 51, which has a smooth outer surface. End 51 can be inserted into a tubular portion 64 of a spigot 60 which screws onto an inlet 72 of a waste container 70. FIG. 11C shows a connector 54, which is either bonded or fitted onto outflow hose 50, and which inserts into an inlet 72 of a waste container 70. Connector 54 is designed with a forward portion 55 and rearward portion 56. Rearward portion 56 adapts to outlet end 51 of the outflow hose 50. Forward portion 55 has an annular expansion 57. Connector 54 easily inserts into inlet 72 to form a sealing engagement. Annular expansion 57 requires that connector 54 be disengaged with some force, so that inadvertent spillage of effluent from waste container 70 is prevented.

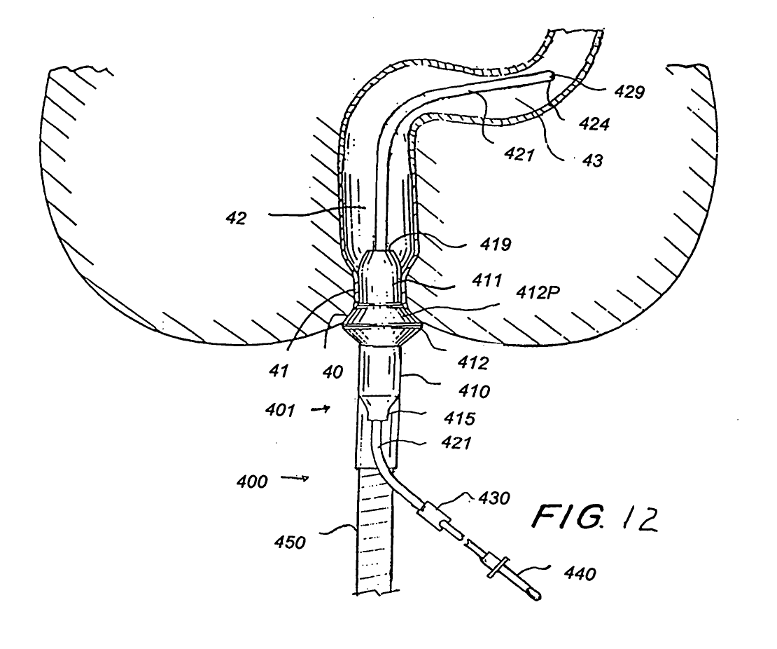

Referring now to FIG. 12, in one exemplifying use of rectocolonic irrigator 400 to lavage the sigmoid colon and rectum, proximal portion 411 of tubular body 410 is inserted into the rectum 42 of an anesthetized patient through the anus 41. Spike 440 is inserted into the spike adapter of a fluid container (not shown). Colonic tube 421 is passed through distal end 415 of tubular body 410, though opening 419 into the rectum 42, and through the rectum 42 so that tip 424 of colonic tube 421 sits in the sigmoid colon 43. Inflow pinch clamp 430 is opened to allow lavage fluid to flow from the fluid container through colonic tube 421, into the sigmoid colon 43. The lavage fluid then washes alongside colonic tube 421 distally through the sigmoid colon 43 into the rectum 42, through opening 419 of tubular body 410, into tubular body 410, through side tube 416 thereof and through outflow tubing 450 into a waste container. Fluid cannot leak from the anus 41 around tubular body 410 because proximal portion 411 of tubular body 410 is dimensioned such that its walls dilate the anus 41 whereby the elastic anus 41 constricts around the proximal portion 411 of tubular body 410 to form a seal between the walls of the proximal portion 411 and the walls of the anus 41. Thus, fluid cannot pass between the outer wall of tubular body 410 and the wall of the anus 41. Surface 412P of expanded portion 412 is pressed against the anal opening 40 to form an additional seal. Lavage is continued until the bowel is sufficiently clean.

Alternatively, colonic tube 421 can be severed at location A-A (FIG. 10A) and the rearward severed end of a proximal portion 421P of colonic tube 421 can be attached by means of a tubing connector and suction tubing to a suction cannister waste receptacle, and residual fluid and waste can be aspirated through aperture 429 (and additional apertures in proximity to tip 424, if present) as proximal portion 421P of colonic tube 421 is withdrawn from the bowel.

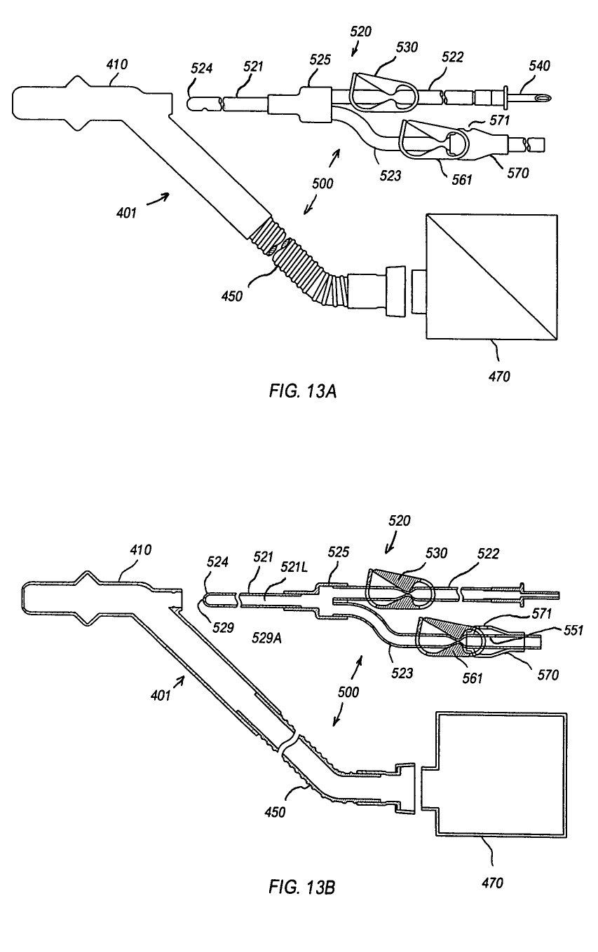

FIGS. 13A and 13B illustrate a fifth embodiment of this invention, rectocolonic irrigator 500, which includes the drain assembly 401, container 470 connected to the outlet end of the tubular body outflow hose 450 (e.g., in any of the ways described above with respect to FIGS. 11A, 11B and 11C), and a colonic tube assembly 520.

Colonic tube assembly 520, comprises a colonic tube 521, an inflow tubing 522, an inflow pinch clamp 530 arranged on inflow tubing 522, a spike 540 attached to an inlet end of inflow tubing 522 and which adapts the inflow tubing 522 to a fluid container (not shown), a colonic tube outflow tubing 523 and a pinch clamp 561 to control colonic tube outflow through colonic tube outflow tubing 523. Colonic tube 521 is joined by means of a Y-connector 525 to inflow tubing 522 and colonic tube outflow tubing 523.

Colonic tube 521 is an elongated tube having an aperture 529 at its tip 524. One or more additional apertures 529A may be placed in the wall of colonic tube 521 in proximity to tip 524. Colonic tube 521 may be fabricated from an elastomeric material compatible for contact with internal parts of the human body, namely the bowel.

Colonic tube outflow tubing 523 is coupled directly to a suction cannister waste receptacle so that effluent can be aspirated from the bowel via the colonic tube 521. Colonic tube outflow tubing pinch clamp 561 may be joined to a suction control locator sleeve 570, having a suction control aperture 571 situated over an aperture 551 in the colonic tube outflow tubing 523 (as similarly illustrated in FIGS. 4A and 4B), so that suction pressure transmitted to the bowel via the colonic tube 521 can be controlled.

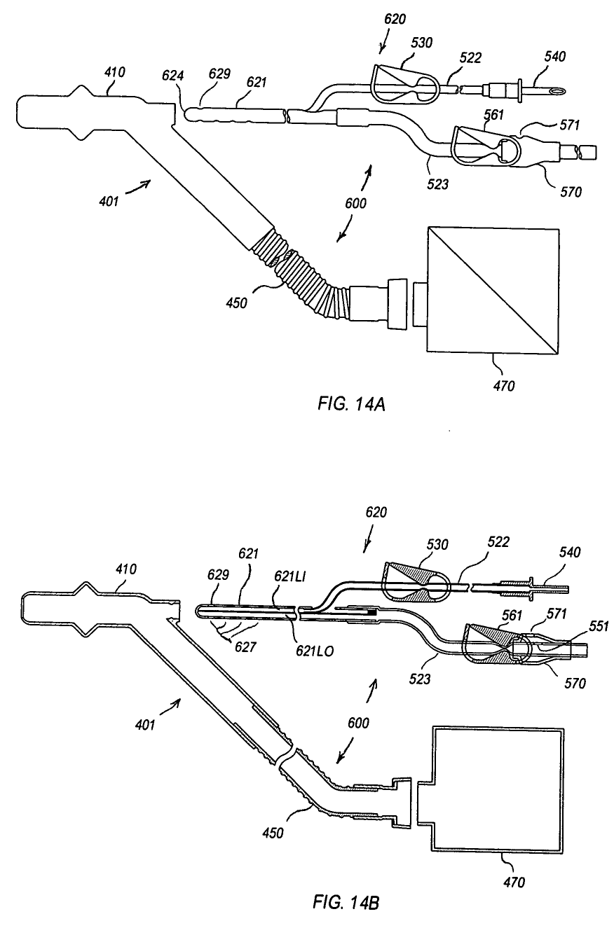

FIGS. 14A and 14B illustrate a sixth embodiment of this invention, rectocolonic irrigator 600, which includes the drain assembly 401 described above, container 470 connected to the outlet end of the tubular body outflow hose 450 (e.g., in any of the ways described above with respect to FIGS. 11A, 11B and 11C), and a colonic tube assembly 620.

Colonic tube assembly 620 is substantially the same as colonic tube assembly 520 except that colonic tube 621 is different than colonic tube 521 and Y-connector 525 is eliminated. Colonic tube 621 comprises two lumens 621LI and 621LO (see FIG. 14B). Inflow tubing 522 inserts into lumen 621LI which is in flow communication with the bowel via an aperture 629 in colonic tube 621. Outflow tubing 523 is joined to the distal end of colonic tube 621 and is in flow communication with lumen 621LO.

When pinch clamp 530 is open and pinch clamp 561 is closed, lavage fluid passes through lumen 621LI of colonic tube 621 and into the bowel through aperture 629.

When pinch clamp 530 is closed and pinch clamp 561 is opened effluent passes through apertures 627, lumen 621LO and colonic tube outflow tubing 523 into the waste receptacle.

As with the fifth embodiment, i.e. rectocolonic irrigator 500, colonic tube outflow tubing 523 is coupled directly to a suction cannister waste receptacle so that effluent can be aspirated from the bowel via the colonic tube 621. Colonic tube outflow tubing pinch clamp 561 may be joined to a suction control locator sleeve 570, having a suction control aperture 571 situated over an aperture 551 in the colonic tube outflow tubing 523 (as similarly illustrated in FIGS. 4A and 4B), so that suction pressure transmitted to the bowel via the colonic tube 621 can be controlled with colonic tube outflow pinch clamp 561 and suction control locator sleeve 570.

Rectocolonic irrigator 600 functions similarly to the rectocolonic irrigator 500 except that irrigation fluid delivered to colonic tube 621 is carried through colonic tube 621 to near its tip 624 in lumen 621LI, and does not mix with effluent, until it enters the bowel through aperture 629. Effluent enters colonic tube 621 through apertures 627 and passes through lumen 621LO, through colonic tube outflow tubing 523 to the suction cannister waste receptacle. The colonic tube assembly 620 of this embodiment prevents the return of effluent fluid caught within the colonic tube 621 back into the bowel.

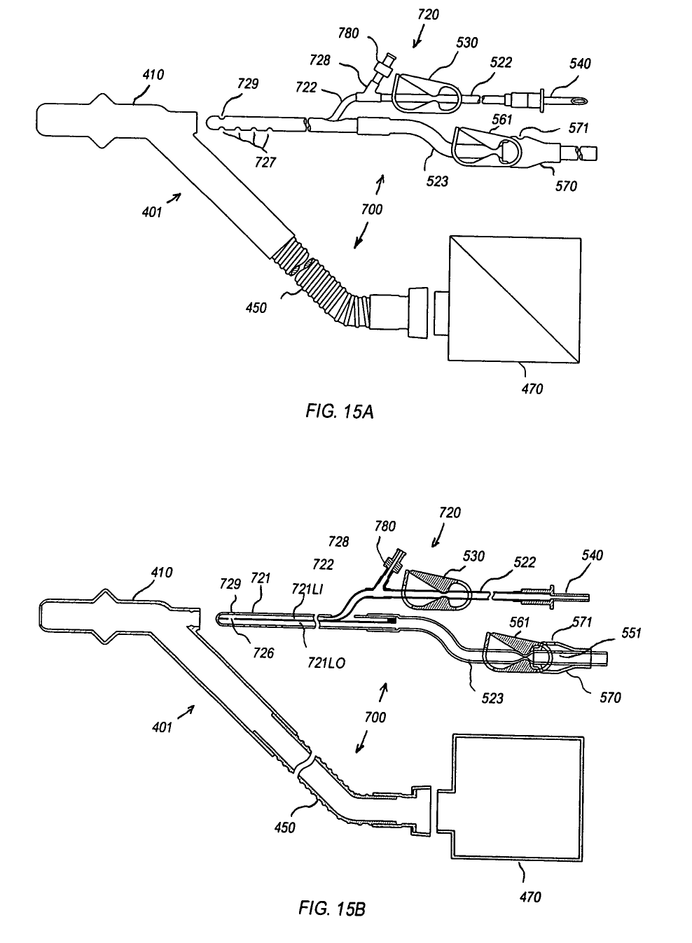

FIGS. 15A and 15B illustrate a seventh embodiment of this invention, rectocolonic irrigator 700, which includes the drain assembly 401 described above, container 470 connected to the outlet end of the tubular body outflow hose 450 (e.g., in any of the ways described above with respect to FIGS. 11A, 11B and 11C), and a colonic tube assembly 720.

Like colonic tube assembly 620, colonic tube assembly 720 includes spike 540, inflow tubing 522 and inflow pinch clamp 530. Colonic tube assembly 720 also includes check valve 780, Y-connector 728 and inflow tube 722. Inflow tubing 522 and check valve 780 join Y-connector 728 which joins inflow tube 722 which inserts into lumen 721LI of colonic tube 721. Colonic tube 721 differs from colonic tube 621 in that colonic tube lumen 721LI communicates directly with colonic lumen 721LO through opening 726 in the wall between the two lumens, in close proximity to tip 724 of colonic tube 721 (see FIG. 15B).

When pinch clamp 530 is open and pinch clamp 561 is closed, lavage fluid passes into the bowel through aperture 729 or by passing through aperture 726 into lumen 721LO and then into the bowel.

When pinch clamp 530 is closed and pinch clamp 561 is opened effluent passes through apertures 727, lumen 721LO and colonic tube outflow tubing 523 into the suction cannister waste receptacle. Effluent may also pass through apertures 729 and 726 into lumen 721LO.

As with the fifth and sixth embodiments, colonic tube outflow tubing 523 is coupled directly to a suction cannister waste receptacle so that effluent can be aspirated from the bowel via the colonic tube 721. A suction control locator sleeve 570 may be arranged in connection with pinch clamp 561 and includes a suction control aperture 571 situated over an aperture 551 in the colonic tube outflow tubing 523 (as similarly illustrated in FIGS. 4A and 4B), so that suction pressure transmitted to the bowel via the colonic tube 721 can be controlled with colonic tube outflow pinch clamp 561 and the suction control locator sleeve 570.

Whereas effluent may be trapped in lumen 621LO of colonic tube 621 of rectocolonic irrigator 600 after the bowel has emptied of all air and fluid in the segment in which the tip 624 lies, because of the vacuum which develops behind the column of effluent within the tube, effluent will not be likewise trapped in lumen 721LO of colonic tube 721 of rectocolonic irrigator 700. Effluent lumen 721LO will discharge completely, because when the bowel in proximity to the tip 724 empties, air is aspirated through check valve 780, inflow tube 722, lumen 721LI and aperture 726 into lumen 721LO, which allows all fluid within the colonic tube 721 to empty via outflow tubing 523.

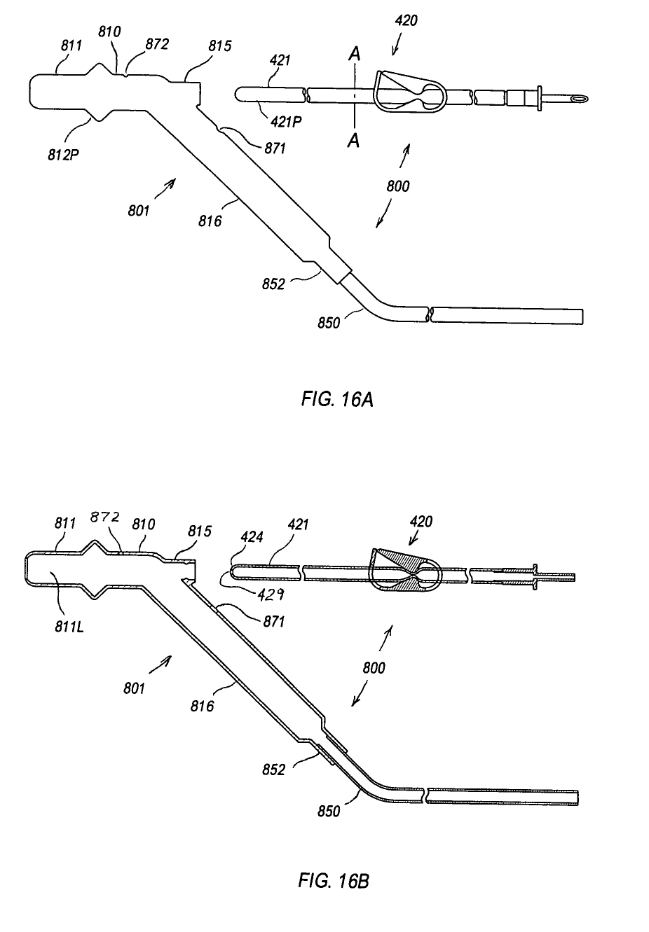

FIGS. 16A and 16B illustrate an eighth embodiment of the invention, rectocolonic irrigator 800, which comprises a drain assembly 801 and colonic tube assembly 420, substantially as described above.

The drain assembly 801 is similar to drain assembly 401 shown FIG. 13A except that it incorporates a suction control aperture 871 in the side tube 816 and substitutes a smaller diameter outflow tubing 850 for the large diameter hose 450. The disposable plastic thin walled waste container 470 is eliminated. Outflow tubing 850, which is attached to a narrowing, outlet 852 of side tube 816, is operatively adaptable to a suction cannister waste receptacle.

Flow into the suction cannister waste receptacle is aided by vacuum pressure. The amount of vacuum pressure transmitted to the interior 811L of proximal portion 811 of tubular body 810 and to the rectum is controlled by the occlusion or uncovering of the suction control aperture 871 in side tube 816 of tubular body 810. When suction control aperture 871 is uncovered, ambient air is drawn into side tube 816 of tubular body 810, diminishing the vacuum pressure therein. When suction control aperture 871 is occluded, vacuum pressure within tubular body 810 increases. Tubular body 810 is similar to tubular body 410 and may be provided with any of its modifications, e.g., the conical distal opening shown in FIG. 10C. However, one difference between drain assembly 801 and drain assembly 401 is that tubular body 810 preferably includes a vacuum relief aperture 872 arranged on the top of the tubular body 810 distal to the expanded portion 812. Vacuum relief aperture 872 is smaller than suction control aperture 871. When suction control aperture 871 is occluded, vacuum relief aperture 872 regulates (i.e., limits) suction pressure being delivered, e.g., to the rectum, and insures that the tubular body 810 does not collapse under the vacuum pressure. Such a vacuum relief aperture in the tubular body can be provided in any of the other embodiments herein wherein the tubular body includes a suction control aperture and vacuum pressure builds up in the tubular body.

The colonic tube assembly 420 is substantially identical to the colonic tube assembly of rectocolonic irrigator 400 (FIGS. 10A and 10B). One possible difference is that instead of a single, relatively large aperture 429 at tip 424, several smaller holes or apertures can be formed at tip 424 to produce a spray at the tip 424 instead of a stream which would be produced when the single, larger aperture 429 is provided. The same modification is applicable to other embodiments of colonic tube assemblies disclosed herein to the extent possible.

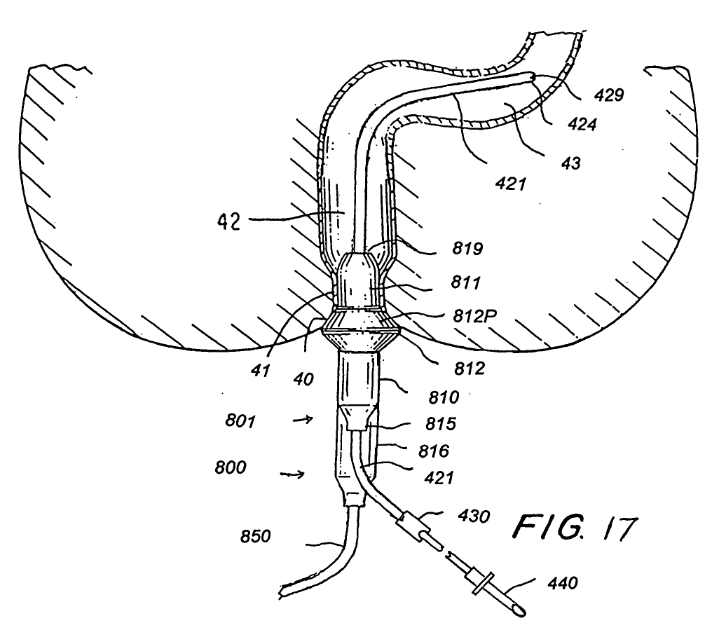

Referring now to FIG. 17, in one exemplifying use of rectocolonic irrigator 800 to lavage the sigmoid colon and rectum, proximal portion 811 of tubular body 810 is inserted into the rectum 42 of an anesthetized patient through the anus 41. Spike 440 is inserted into the spike adapter of a fluid container (not shown). Colonic tube 421 is passed through distal end 815 of tubular body 810, though opening 819 into the rectum 42, and through the rectum 42 so that tip 424 of colonic tube 421 lies in the sigmoid colon 43. Inflow pinch clamp 430 is opened to allow lavage fluid to flow from the fluid container through colonic tube 421, into the sigmoid colon 43. The lavage fluid then washes alongside colonic tube 421 distally through the sigmoid colon 43 into the rectum 42, through opening 819 of tubular body 810, into tubular body 810, through side tube 816 thereof and through outflow tubing 850 into a suction cannister waste receptacle. Fluid cannot leak from the anus 41 around tubular body 810 because proximal portion 811 of tubular body 810 is dimensioned such that its walls dilate the anus 41 whereby the elastic anus 41 constricts around the proximal portion 811 of tubular body 810 to form a seal between the walls of the proximal portion 811 and the walls of the anus 41. Thus, fluid cannot pass between the outer wall of tubular body 810 and the wall of the anus 41. Surface 812P of expanded portion 812 is pressed against the anal opening 40 to form an additional seal. Lavage is continued until the bowel is sufficiently clean.

When suction control aperture 871 (see FIG. 16B) in side tube 816 is open (uncovered), ambient air is aspirated through aperture 871, thereby limiting the vacuum pressure transmitted to interior space 811L of proximal portion 811 of tubular body 810 and the rectum 42. Lavage effluent and fecal waste are forced into tubular body 810 and into side tube 816 primarily by gravity and elevated pressure in the rectum 42. Transmitting vacuum pressure to the rectum 42 is not desired at this point, because the vacuum pressure could result in rectal tissue being sucked into the opening 819 of tubular body 810, thereby occluding opening 819, interfering with rectal emptying. However, when the rectum 42 has largely emptied, there is no longer pressure on the fluid in the rectum 42. Fluid pressure and gravity are not able to empty the bowel of all residual fluid and fecal waste. Now it may be useful to occlude suction control aperture 871 in side tube 816 to allow vacuum pressure to be transmitted through tubular body 810 to the rectum 42 to aspirate the residual fluid.

Alternatively, colonic tube 421 can be severed at location A-A (FIG. 16A) and the rearward severed end of a proximal portion 421P of colonic tube 421 can be attached by means of a tubing connector and suction tubing to a suction cannister waste receptacle, and residual fluid and waste can be aspirated through aperture 429 (and additional apertures in proximity to tip 424, if present) as proximal portion 421P of colonic tube 421 is withdrawn from the bowel. This same modification applies to the embodiment shown in FIG. 10B.

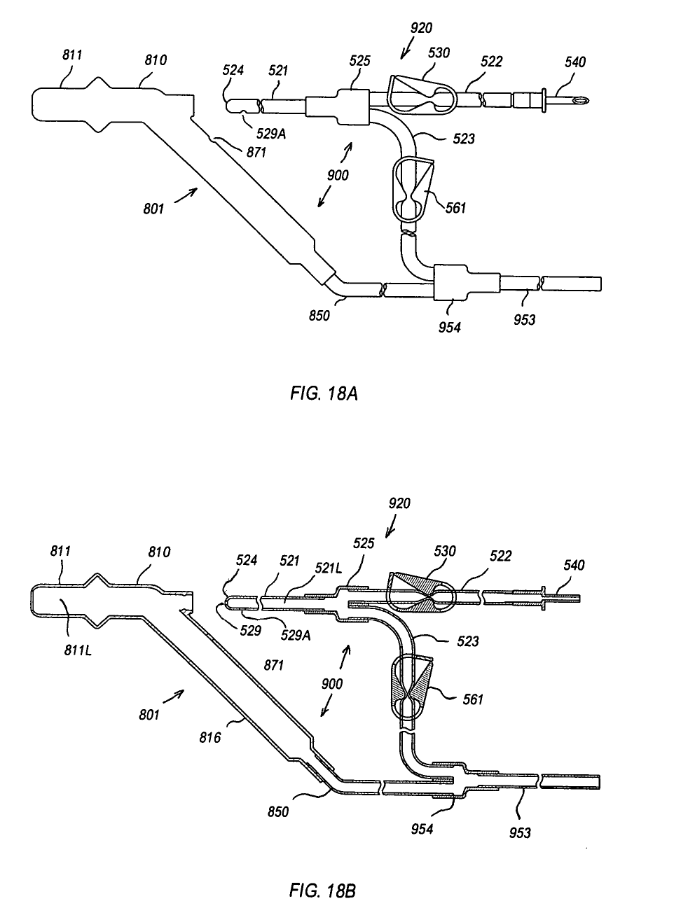

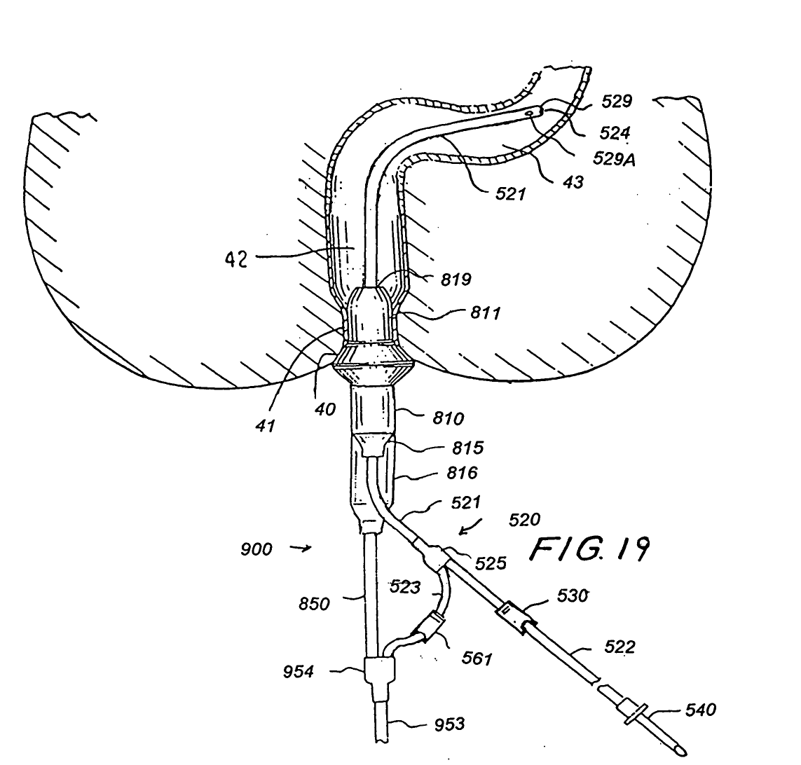

FIGS. 18A and 18B, illustrate a ninth embodiment of the invention, rectocolonic irrigator 900, which comprises drain assembly 801, described above, a colonic tube assembly 920, Y-connector 954 and rectocolonic irrigator outflow tubing 953.

Colonic tube assembly 920, is substantially the same as colonic tube assembly 520 (FIGS. 13A and 13B), except that outflow tubing 523 is connected by means of Y-connector 954 to rectocolonic irrigator outflow tubing 953, which operatively adapts to a port of a suction cannister waste receptacle (not shown), and suction control locator sleeve 570 and outflow tubing aperture 551 are eliminated.

When pinch clamp 530 is open and pinch clamp 561 is closed, lavage fluid passes through lumen 521L of colonic tube 521 and into the bowel through apertures 529 and 529A.

When pinch clamp 530 is closed and pinch clamp 561 is opened effluent passes through apertures 529 and 529A, lumen 521L, colonic tube outflow tubing 523 and rectocolonic irrigator outflow tubing 953 into the waste receptacle.

Flow into the suction cannister waste receptacle is aided by vacuum pressure. The amount of vacuum pressure transmitted to an interior 811L of a proximal portion 811 of tubular body 810 and to the rectum, and to a lumen 521L of colonic tube 521 and to the portion of the colon surrounding tip 524 of the colonic tube 521 is controlled by the occlusion or uncovering of suction control aperture 871 in side tube 816 of tubular body 810 (see FIG. 18B).

Referring now to FIG. 19, in one exemplifying use of rectocolonic irrigator 900 to lavage the sigmoid colon 43 and rectum 42, the proximal portion 811 of tubular body 810 of the rectocolonic irrigator 900 is inserted through the anus 41 of an anesthetized patient into the rectum 42. Spike 540 is inserted into the spike adapter of a fluid container. The colonic tube 521 is passed through a distal end 815 of tubular body 810, though opening 819 at the front end of proximal portion 811 into the rectum 42, and through the rectum 42 so that tip 524 of colonic tube 521 lies in the sigmoid colon 43. The colonic tube outflow tubing 523 is occluded by closing pinch clamp 561. Inflow pinch clamp 530 is opened to allow lavage fluid to flow from the fluid container through inflow tubing 522 of colonic tube assembly 520, Y-connector 525 and through colonic tube 521 into the sigmoid colon 43. The lavage fluid then washes distally alongside colonic tube 521, distally through the sigmoid colon 43 into the rectum 42, through opening 819 of tubular body 810, through side tube 816 of tubular body 810, through tubular body outflow tubing 850, through Y-connector 954, and through rectocolonic irrigator outflow tubing 953 into the suction cannister waste receptacle. As discussed above, fluid cannot leak from the anus 41 around tubular body 810 in view of the construction of proximal portion 811 of tubular body 810. Fluid does not leak out of tubular body 810 through suction control aperture 871 since suction is being applied and draws ambient air in through suction control aperture 871 when it is uncovered.

When lavage is nearly complete, inflow tubing pinch clamp 530 is closed and colonic tube outflow tubing pinch clamp 561 is opened. Suction control aperture 871 in side tube 816 of tubular body 810 is occluded, which allows suction pressure to be transmitted through tubular body 810 to the rectum 42 and through colonic tube outflow tubing 523, Y-connector 525 and colonic tube 521 to the sigmoid colon 43. Residual fluid in the rectum 42 is aspirated through opening 819 into tubular body 810, through side tube 816 thereof, through tubular body outflow tubing 850, through Y-connector 954 and through rectocolonic irrigator outflow tubing 953 into the suction cannister. Residual fluid in the sigmoid colon 43 is aspirated through apertures 529 and 529A, through colonic tube 521, through Y-connector 525, through colonic tube outflow tubing 523, through Y-connector 954 and through rectocolonic irrigator outflow tubing 953 into the suction cannister waste receptacle. Suction control aperture 871 is then uncovered, colonic tube 521 is partially withdrawn and aperture 871 is occluded again to aspirate residual fluid from the bowel. This sequence is then repeated until the colonic tube 521 is removed and all residual fluid aspirated.

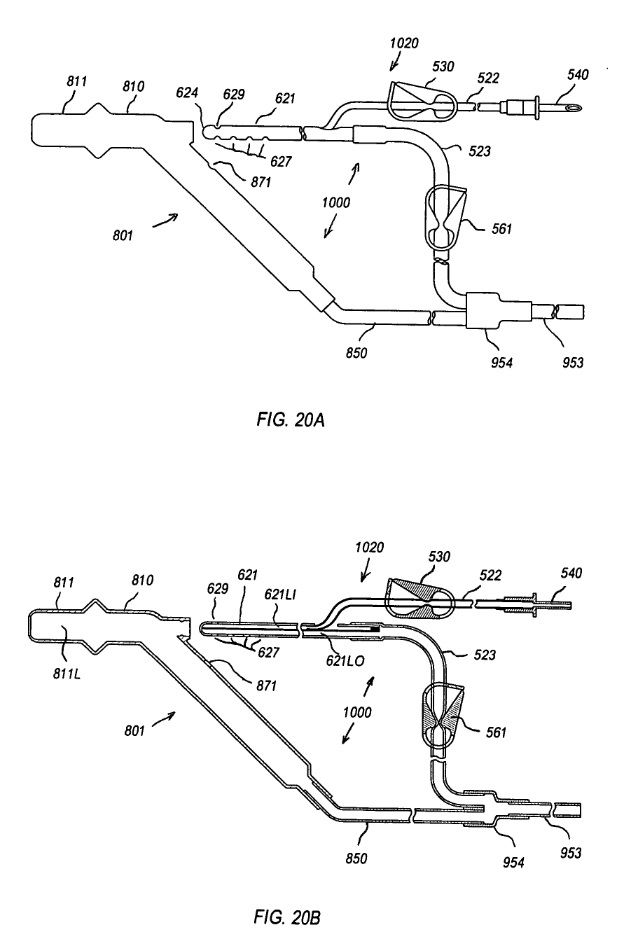

FIGS. 20A and 20B illustrate a tenth embodiment of the invention, rectocolonic irrigator 1000, which comprises drain assembly 801, a colonic tube assembly 1020, Y-connector 954 and rectocolonic irrigator outflow tubing 953.

Colonic tube assembly 1020 is substantially the same as colonic tube assembly 620 (FIGS. 14A and 14B), except that outflow tubing 523 is connected by means of Y-connector 954 to rectocolonic irrigator outflow tubing 953, which operatively adapts to a port of a suction cannister waste receptacle (not shown), and suction control locator sleeve 570 and outflow tubing aperture 551 are eliminated.

When pinch clamp 530 is open and pinch clamp 561 is closed, lavage fluid passes through lumen 621LI of colonic tube 621 and into the bowel through aperture 629.

When pinch clamp 530 is closed and pinch clamp 561 is opened effluent passes through apertures 627, lumen 621LO, colonic tube outflow tubing 523 and rectocolonic irrigator outflow tubing 953 into the waste receptacle.

As with the eighth and ninth embodiments of this invention, i.e., rectocolonic irrigators 800 and 900, flow of effluent into the suction cannister waste receptacle is aided by vacuum pressure. The amount of vacuum pressure transmitted to the interior 811L of proximal portion 811 of tubular body 810 and to the rectum, and to lumen 621LO of colonic tube 621 and to the portion of the colon surrounding the tip 624 of colonic tube 621 is controlled by the occluding or uncovering suction control aperture 871 in side tube 816 of tubular body 810.

Rectocolonic irrigator 1000 functions similarly to the rectocolonic irrigator 900 except that irrigation fluid delivered to colonic tube 621 is carried through colonic tube 621 to near its tip 624 in lumen 621LI, and does not mix with effluent, until it enters the bowel through aperture 629. Effluent enters colonic tube 621 through apertures 627 and passes through lumen 621LO, through colonic tube outflow tubing 523 and rectocolonic irrigator outflow tubing 953 to the suction cannister waste receptacle. The colonic tube assembly 1020 of this embodiment prevents the return of effluent fluid caught within the colonic tube 621 back into the bowel.

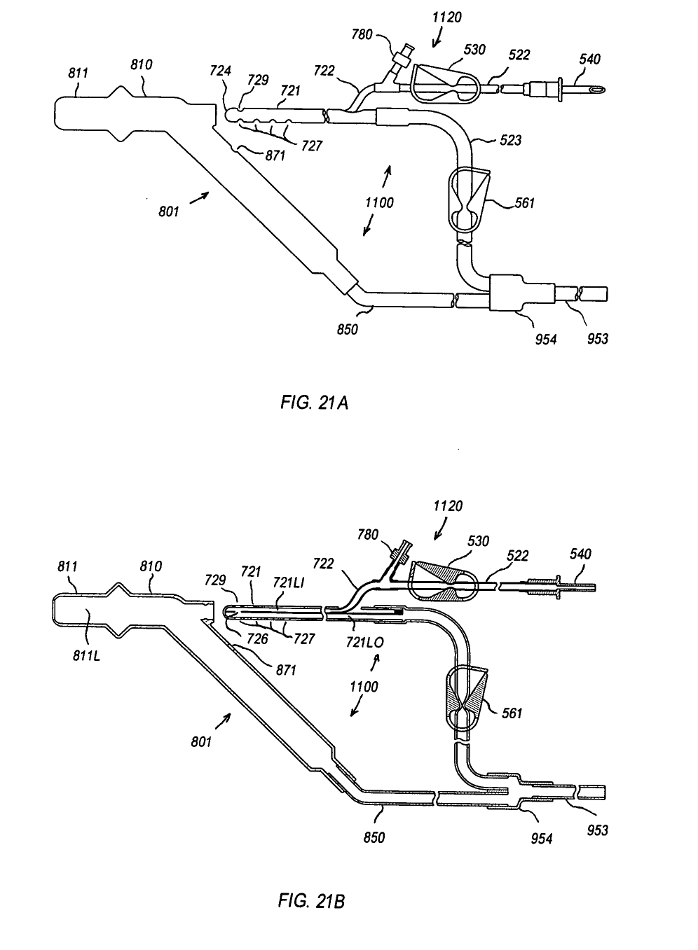

FIGS. 21A and 21B illustrate an eleventh embodiment of the invention, rectocolonic irrigator 1100, which comprises drain assembly 801, a colonic tube assembly 1120, Y-connector 954 and rectocolonic irrigator outflow tubing 953.

Colonic tube assembly 1120 is substantially the same as colonic tube assembly 720 (FIGS. 15A and 15B), except that outflow tubing 523 is connected by means of Y-connector 954 to rectocolonic irrigator outflow tubing 953, which operatively adapts to a port of a suction cannister waste receptacle (not shown), and suction control sleeve 570 and tubing aperture 551 are eliminated.

When pinch clamp 530 is open and pinch clamp 561 is closed, lavage fluid passes through lumen 721LI of colonic tube 721 into the bowel through aperture 729 or by passing through aperture 726 into lumen 721LO and through apertures 727 into the bowel.

When pinch clamp 530 is closed and pinch clamp 561 is opened effluent passes through apertures 727, lumen 721LO, colonic tube outflow tubing 523 and rectocolonic irrigator outflow tubing 953 into the waste receptacle. Effluent may also pass through apertures 729 and 726 into lumen 721LO.

As with the eighth, ninth and tenth embodiments of this invention, i.e., rectocolonic irrigators 800, 900 and 1000, flow of effluent into the suction cannister waste receptacle is aided by vacuum pressure. The amount of vacuum pressure transmitted to the interior 811L of proximal portion 811 of tubular body 810 and to the rectum, and to lumen 721LO of colonic tube 721 and to the portion of the colon surrounding the tip 724 of colonic tube 721 is controlled by the occluding or uncovering suction control aperture 871 in side tube 816 of tubular body 810.

Whereas effluent may be trapped in lumen 621LO of colonic tube 1020 of rectocolonic irrigator 1000 after the bowel has emptied of all air and fluid in the segment in which the tip 624 lies, because of the vacuum which develops behind the column of effluent within the tube, effluent will not be likewise trapped in lumen 721LO of colonic tube 721 of rectocolonic irrigator 1100. Effluent lumen 721LO will discharge completely, because when the bowel in proximity to the tip 724 empties, air is aspirated through check valve 780, inflow tube 722A, lumen 721LI and aperture 726 into lumen 721LO, which allows all fluid within the colonic tube 721 to empty via outflow tubing 523 and rectocolonic irrigator outflow tubing 953.

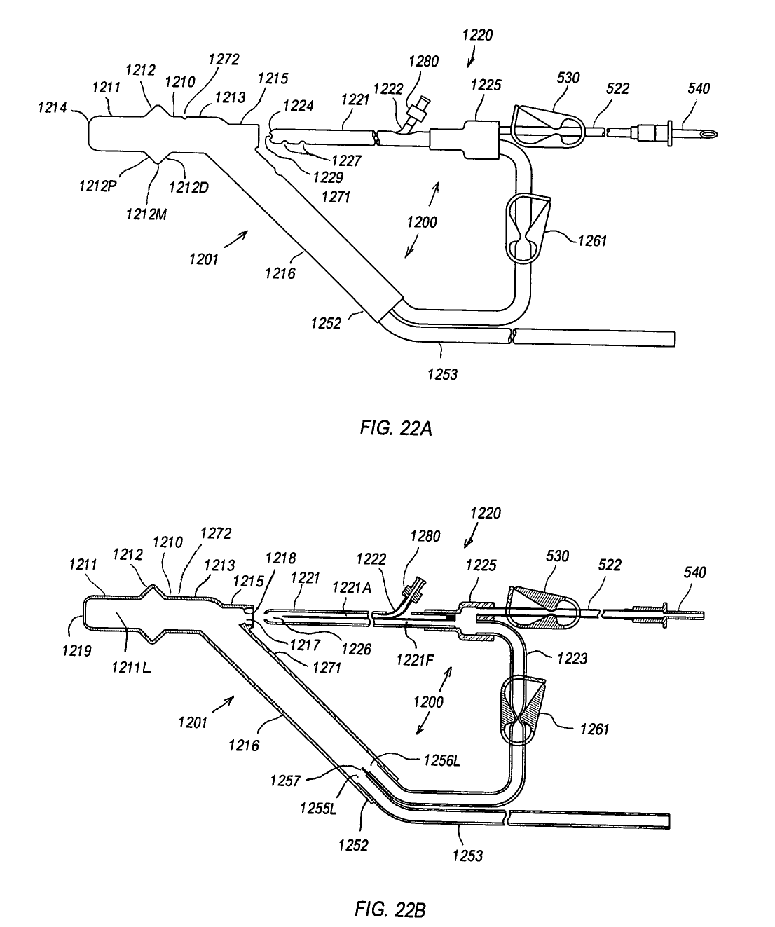

FIGS. 22A and 22B illustrate a twelfth embodiment of the invention, rectocolonic irrigator 1200, which comprises a drain assembly 1201 and a colonic tube assembly 1220.

Drain assembly 1201 is similar to drain assembly 801 shown FIG. 16A and 16B except that distal end 1252 of side tube 1216 comprises two circular lumens 1255L and 1256L separated by septum 1257 (see FIG. 22B). Rectocolonic irrigator outflow tubing 1253 is inserted into lumen 1255L of distal end 1252 of side tube 1216. Further, tubular body 1210 is similar to tubular body 410 and may be provided with any of its modifications, e.g., the conical distal opening shown in FIG. 10C.

Colonic tube assembly 1220 comprises a double lumen colonic tube 1221 with opening 1229 at its proximal tip 1224. The two lumens 1221A and 1221 F communicate with each other through a defect in the septum to provide a common lumen 1226 near the tip 1224 of the colonic tube 1221. Inflow tubing 522 and colonic tube outflow tubing 1223 are both in flow communication with colonic tube lumen 1221F through Y-connector 1225. Check valve 1280 is in flow communication with colonic tube lumen 1221A via connector tubing 1222 (see FIG. 22B).

When pinch clamp 530 is open and pinch clamp 1261 is closed, lavage fluid passes into the bowel through inflow tubing 522, Y-connector 1225, lumen 1221 F and apertures 1227 into the bowel and/or from lumen 1221 F through common lumen 1226 through opening 1229 into the bowel.

When pinch clamp 530 is closed and pinch clamp 1261 is opened, effluent can flow through apertures 1229 and 1227, lumen 1221F, colonic tube outflow tubing 1223 and rectocolonic irrigator outflow tubing 1253 into the waste receptacle.

As with the eighth, ninth, tenth and eleventh embodiments of this invention, i.e., rectocolonic irrigators 800, 900, 1000 and 1100, flow of effluent into a suction cannister waste receptacle is aided by vacuum pressure. The amount of vacuum pressure transmitted to the interior space 1211L of proximal portion 1211 of tubular body 1210 and to the rectum and to lumen 1221F of colonic tube 1221 and to the portion of the colon surrounding the tip 1224 of colonic tube 1221 is controlled by the occlusion or uncovering of a suction control aperture 1271 in side tube 1216 of tubular body 1210. When suction control aperture 1271 is uncovered, ambient air is drawn into side tube 1216 of tubular body 1210, diminishing the vacuum pressure therein. When suction control aperture 1271 is occluded, vacuum pressure within tubular body 1210 increases. However, when suction control aperture 1271 is occluded during use, vacuum relief aperture 1272 limits suction pressure being delivered, e.g., to the rectum, and insures that the tubular body 1210 does not collapse under the vacuum pressure.

Whereas effluent may be trapped in lumen 521L of colonic tube 521 of rectocolonic irrigator 900 (see FIG. 18B) after the bowel has emptied of all air and fluid in the segment in which the tip 524 lies, because of the vacuum which develops behind the column of effluent within the colonic tube 521, effluent will not be likewise trapped in lumen 1221F of colonic tube 1221 of rectocolonic irrigator 1200. Effluent lumen 1221F will discharge completely, because when the bowel in proximity to the tip 1224 empties, air is aspirated through check valve 1280, connecting tube 1222, lumen 1221A and through common lumen 1226 into lumen 1221F, which allows all fluid within the colonic tube 1221 to empty via colonic tube outflow tubing 1223 and rectocolonic irrigation outflow tubing 1253.

With respect to the above-described rectocolonic irrigator 1100, one advantage that rectocolonic irrigator 1200 has over rectocolonic irrigator 1100 is that inflow flow rates may be higher, without comprising outflow, since when using rectocolonic irrigator 1100, outflow lumen 1121LO of colonic tube 1121 is larger than inflow lumen 1121LI whereas fluid lumen 1221F of colonic tube 1221 (through which lavage fluid passes into the bowel) is larger than air lumen 1221A. Another advantage is that construction is simpler by incorporating the function of Y-connector 954 into tubular body 1210.

With respect to the configuration of the various outflow hoses and tubings described in the embodiments above, the wide diameter outflow hose 450 may have a straight tubular, spiral or concertina configuration. Spiral and concertina configurations allow the outflow hose 450 to be thin walled and flexible but collapse resistant. The smaller diameter outflow tubings 150, 350, 850, 953 and 1250 may also have a straight tubular, spiral or concertina configuration. Described above are several irrigator devices, drain assemblies, colonic tubes and colonic tube assemblies which include the colonic tubes. Each of these irrigator devices, drain assemblies, colonic tubes and colonic tube assemblies can be considered a separate invention and the use thereof as described above is not limiting. Rather, for example, it is envisioned that the various colonic tubes can be used in different ways than as described above and possibly even in procedures other than those described above. Moreover, each of the colonic tube assemblies described above can be used with a different irrigator device and/or with a different drain assembly, e.g., an irrigator device or drain assembly including a tubular body without an expanded portion.

In any of the embodiments above where a colonic tube is passed through a tubular body, instead of providing a single opening at the proximal end of the tubular body through which the colonic tube passes into the colon and lavage fluid enters after washing distally through the colon into the rectum, it is possible to provide the proximal portion of the tubular body with a port which provides a sliding or sealing fit for passage of only the colonic tube therethrough and form one or more drain holes on the proximal portion leading to the interior of the tubular body. In this case, the lavage fluid passes through these drain holes and does not pass through the same opening at the proximal end of the tubular body as the colonic tube.

While particular embodiments of the invention have been shown and described, it will be obvious to those skilled in the art that changes and modifications may be made without departing from the invention in its broader aspects, and, therefore, the aim in the appended claims is to cover all such changes and modifications as fall within the true spirit and scope of the invention.

1. A colorectal irrigator device, comprising:
a tubular body defining an interior space and having a proximal portion adapted to be inserted into an anus of a person such that said proximal portion causes the anus to constrict around said proximal portion and thereby seal said proximal portion against the anal wall and a distal portion,
said proximal portion including an opening at a tip thereof,
said distal portion including a distal end having an opening in line with said opening in said proximal portion and a side tube arranged at an angle to said proximal portion, said distal end being adapted to accommodate a colonic tube passing through said opening thereof and said side tube being adapted to mate with an outflow tube.

2. The device of claim 1, wherein said tubular body further includes an expanded portion having a larger size than said proximal portion and interposed between said proximal portion and said distal portion, said expanded portion being adapted to engage with the anal opening to limit insertion of said proximal portion into the anus and seal said body against the anal opening.

3. The device of claim 2, wherein said proximal portion includes a circumferential lip which is an expanded portion having a smaller diameter than said expanded portion, said lip being adapted to retain said proximal portion in the anorectum and help to prevent inadvertent expulsion of the device.

4. The device of claim 1, wherein said distal end includes an integral seal adapted to sealingly engage with the colonic tube to prevent fluid from escaping through said distal end around the colonic tube.

5. The device of claim 1, wherein said side tube has a rear end narrower than a remaining portion of said side tube.

6. The device of claim 1, wherein said side tube includes a suction control aperture arranged to control vacuum pressure transmitted to said tubular body.

7. The device of claim 6, wherein said side tube further includes a vacuum relief aperture arranged to regulate vacuum pressure in said tubular body.

8. A colorectal lavage device for use in cleansing colon and the rectum, comprising:
the device of claim 1;
a colonic tube assembly including a colonic tube arranged to pass through said opening in said distal end of said tubular body, said colonic tube having a tip and at least one aperture at or near said tip;
a tubular body outflow tubing having an inlet end portion coupled to said side tube;
inflow control means for controlling inflow of fluid through said colonic tube.

9. The device of claim 8, wherein said inflow control means comprises a pinch clamp arranged to pinch said colonic tube.

10. The device of claim 8, wherein said colonic tube assembly further comprises
a first Y-connector;
an inflow tubing having an inlet end connectable to a lavage fluid container and an outlet end coupled to said first Y-connector, said inflow control means being arranged in connection with said inflow tubing;
a second Y-connector;
a colonic tube outflow tubing having an inlet end connected to said first Y-connector and an outlet end connected to said second Y-connector;
outflow control means arranged in connection with said colonic tube outflow tubing for controlling outflow of effluent through said colonic tube outflow tubing, said colonic tube being joined by means of said first Y-connector to said inflow tubing and said colonic tube outflow tubing; and
a rectocolonic irrigator outflow tubing having an outlet end connectable to a vacuum pressure source, said colonic tube outflow tubing and said tubular body outflow tubing being joined by said second Y-connector to an inlet end of said rectocolonic irrigator outflow tubing.

11. The device of claim 10, wherein said colonic tube has at least one additional aperture in proximity to said tip.

12. The device of claim 8, wherein said colonic tube comprises first and second lumens, a first aperture in communication with said first lumen and at least one second aperture in communication with said second lumen, said colonic tube assembly further comprising:
an inflow tubing having an inlet end connectable to a lavage fluid container and an outlet end arranged in said first lumen, said inflow control means being arranged in connection with said inflow tubing;
a Y-connector;
a colonic tube outflow tubing having an inlet end coupled to said second lumen and an outlet end connected to said Y-connector;
outflow control means arranged in connection with said colonic tube outflow tubing for controlling outflow of effluent through said colonic tube outflow tubing; and
a rectocolonic irrigator outflow tubing having an outlet end connectable to a vacuum pressure source, said colonic tube outflow tubing and said tubular body outflow tubing being joined by said Y-connector to an inlet end of said rectocolonic irrigator outflow tubing.

13. The device of claim 8, wherein said colonic tube comprises first and second lumens, a first aperture in communication with said first lumen, at least one second aperture in communication with said second lumen, and an additional aperture opening between said first and second lumens proximate said tip of said colonic tube, said colonic tube assembly further comprising:
a first Y-connector;
a first inflow tubing having an inlet end connectable to a lavage fluid container and an outlet end connected to said first Y-connector, said inflow control means being arranged in connection with said inflow tubing;
a check valve;
a second inflow tubing coupling said first check valve to said first lumen of said colonic tube;
a second Y-connector;
a colonic tube outflow tubing having an inlet end coupled to said second lumen of said colonic tube and an outlet end connected to said second Y-connector;
outflow control means arranged in connection with said colonic tube outflow tubing for controlling outflow of effluent through said colonic tube outflow tubing; and
a rectocolonic irrigator outflow tubing having an outlet end connectable to a vacuum pressure source, said colonic tube outflow tubing and said tubular body outflow tubing being joined by said second Y-connector to an inlet end of said rectocolonic irrigator outflow tubing.

14. The device of claim 8, wherein said side tube has a distal end having first and second substantially circular lumens, and a septum for separating said first and second lumens, said tubular body outflow tubing being connected to said first lumen, said colonic tube assembly further comprising
a Y-connector having a single lumen front end and a double lumen rear end, said colonic tube being connected to said lumen at said front end of said Y-connector;
an inflow tubing having an inlet end connectable to a lavage fluid container and an outlet end coupled to one of said lumens at said rear end of said Y-connector, said inflow control means being arranged in connection with said inflow tubing;
a colonic tube outflow tubing having an inlet end connected to the other of said lumens at said rear end of said Y-connector and an outlet end connected to said second lumen at said distal end of said side tube; and
outflow control means arranged in connection with said colonic tube outflow tubing for controlling outflow of effluent through said colonic tube outflow tubing,
said tubular body outflow tubing constituting a rectocolonic irrigator outflow tubing.

15. The device of claim 14, wherein said colonic tube comprises first and second lumens, a septum separating said first and second lumens to define a common lumen adjacent said aperture at or near said tip, and at least one aperture in communication with said first lumen, said first lumen of said colonic tube being in communication with said inflow tubing and said colonic tube outflow tubing via said Y-connector, said colonic tube assembly further comprising:
a check valve in flow communication with said second lumen of said colonic tube.

16. The device of claim 8, wherein said colonic tube comprises first and second lumens, a septum separating said first and second lumens to define a common lumen adjacent said aperture at or near said tip, and at least one aperture in communication with said first lumen, said colonic tube assembly further comprising:
an inflow tubing system having an inlet coupled to a lavage fluid container and an outlet coupled to said first lumen of said colonic tube, said inflow control means being arranged in connection with said inflow tubing system; and
a check valve in flow communication with said second lumen of said colonic tube.

17. The device of claim 8, further comprising a spike attached to an inlet end of said colonic tube for connecting said colonic tube to a lavage fluid container.

18. The device of claim 8, wherein said colonic tube has a plurality of arms, further comprising a plurality of spikes, each of said spikes being attached to an inlet end of a respective one of said arms of said colonic tube for connecting said arm to a respective fluid container.

19. The device of claim 8, wherein an inlet end of said colonic tube is integrally attached to a lavage fluid container.

20. The device of claim 8, wherein said colonic tube has a plurality of arms, each of said arms being integrally connected to a respective fluid container.
